(19) United States
(12) Patent Application Publication
(10) Pub. No.: US 2020/0261580 A1
(43) Pub. Date: Aug. 20, 2020

(54) Photodynamic Therapy Method for Skin Disorders

(71) Applicant: Andrea Willey, Sacramento, CA (US)

(72) Inventor: Andrea Willey, Sacramento, CA (US)

(21) Appl. No.: 16/631,205

(22) PCT Filed: Jul. 17, 2018
(86) PCT No.: PCT/US18/42505

§ 371 Date: Jan. 15, 2020

Related U.S. Application Data
(60) Provisional application No. 62/533,558, filed on Jul. 17, 2017.
(60) Provisional application No. 62/534,973, filed on Jul. 20, 2017.

(51) Int. Cl. A61K 41/00; A61K 31/197; A61K 9/00; A61K 9/06; A61K 9/107; A61P 17/10

(52) U.S. Cl. CPC A61K 41/0052; A61K 31/197; A61P 17/10; A61K 9/06; A61K 9/1075; A61K 9/0014

(57) ABSTRACT

The present invention is directed to methods of treating diseases and disorders of the skin (e.g., acne) with heat-enabled photodynamic therapy (HEPT). Methods of treating acne, non-melanoma skin cancer (NMSC), actinic keratosis (AK) or disseminated superficial actinic porokeratosis (DSAP) using red light photodynamic therapy on heat-treated skin.

### CROSS REFERENCE TO RELATED APPLICATIONS

This application claims the benefit of U.S. Provisional Patent Application 62/533,558, filed Jul. 17, 2017 and U.S. Provisional Patent Application 62/534,973, filed Jul. 20, 2017, the contents of which are herein incorporated by reference in their entirety.

### FIELD OF THE INVENTION

The present invention relates generally to methods of treating diseases and disorders of the skin using red light photodynamic therapy in conjunction with heating. In particular embodiments, the present invention relates to methods of treating acne, non-melanoma skin cancer (NMSC), actinic keratosis (AK) or disseminated superficial actinic porokeratosis (DSAP) using red light photodynamic therapy on heat-treated skin.

### BACKGROUND OF THE INVENTION

Skin disease is among the most common human illnesses, affecting between 30% and 70% of the population, with even higher rates in at-risk subpopulations. (Hay R J et al., J Invest Dermatol. 2014. 134(6): 1527-1534). The healthcare burden is equally significant, estimated at nearly $27 direct health care costs and an addition 11 billion in lost productivity. (Lim H W et al. J Am Acad Dermatol. 2017; 76: 958-972).

Acne is the most common skin disease. Non-inflammatory acne is the most common type, characterized by whiteheads and blackheads. Inflammatory acne typically involves a bacterial (P. acnes) infection and is characterized by papules, pustules, nodules, and cysts. The most common trigger for both types of acne is hormonal, with most individuals experiencing at least a mild form of acne in adolescence. Other triggers may include diet, stress and certain medications. Because acne affects the appearance, and often at a critical age, it can negatively impact the quality of life. (Kligman A M. J Invest Dermatol. 1974; 62:268-87).

Various methods for treating acne are known, including topical agents such as benzoyl peroxide, retinoids and antiobiotics. (Thiboutot D, et al. J Am Acad Dermatol. 2009; 60(Suppl 5): S1-50). Systemic agents may be used as well, including antibiotics, retinoids and hormone therapy. Yet, many of these approaches have undesirable side effects and/or fail to produce an acceptable result. Antibiotic resistance is increasing, with many countries reporting that more than 50% of P. acnes strains are resistant to particular topical agents.

Ultraviolet (UV)/blue light is approved by the FDA for the treatment of mild to moderate acne, due to its anti-inflammatory effects mediated on skin cells. A procedure combining a photosensitive agent and high intensity red light has been demonstrated to be effective, but with significant side effects. (Hongcharu W, et al., J Invest Dermatol. 2000 August; 115(2):183-92), Sakamoto F H et al., J Am Acad Dermatol. 2010 August; 63(2):183-93); (Sakamoto F H et al. J Am Acad Dermatol. 2010 August; 63(2):195-211). As a result, this approach has been reserved for the most serious cases of acne.

There remains a need for novel approaches to treatment of skin diseases and disorders, including acne but not limited to acne, which provide improved results and limits side effects.

### SUMMARY OF THE INVENTION

The present invention provides a method of treating skin diseases or disorders using red light photodynamic therapy on pre-heated skin.

In embodiment first aspect, the present invention is a method of treating facial acne in a subject in need thereof, comprising (i) applying heat to an affected area of the subject's skin using a thermal delivery device to achieve a skin temperature of between about 38 and about 42° C. for a suitable time; (ii) incubating a pharmaceutical composition comprising a photoactive agent for a period of less than about 14 hours to provide an incubated pharmaceutical composition; (iii) applying a therapeutically effective amount of the incubated pharmaceutical composition to the affected area; (iv) administering a suitable dose of red light to the affected area, therapy treating the facial acne.

In one embodiment, the acne is mild acne, moderate acne or severe acne.

In another embodiment, the acne is comedonal, papulopustular or nodulocystic.

In one embodiment, the thermal delivery device is a heat mask. In a particular embodiment, the thermal delivery device is an acetate mask that heats upon crystallization.

In one embodiment, the affected area is heated for about 60 minutes.

In a particular embodiment, the affected area is heated to about 40° C.

In a particular embodiment, the incubation period is less than about 14 hours but achieves the same effect as if the pharmaceutical composition was incubated for about 14 hours in the absence of step (i).

In a particular embodiment, the incubation period is less than about 3 hours but achieves the same effect as if the pharmaceutical composition was incubated for about 3 hours in the absence of step (i).

In a particular embodiment, the incubation period is less than about 1 hour but achieves the same effect as if the pharmaceutical composition was incubated for about 1 hour in the absence of step (i).

In an exemplary embodiment, the incubation period is less than about 10, less than about 5 or less than about 1 minute.

In one embodiment, the pharmaceutical composition is a nanoemulsion comprising 10% 5-aminolevulinic acid HCl.

In another embodiment, the light has a wavelength of between about 620 to about 640 nm, and more particularly, about 630 nm.

In one embodiment, the suitable dose of light is about 37 J/cm².

In exemplary embodiments, the treatment results in a reduction in the subject's acne lesion count. In a particular embodiment, the reduction persists for a period of at least three months.

In exemplary embodiments, treatment results in a reduction in the subject's acne lesion severity. In a particular embodiment, the reduction persists for a period of at least three months.

In exemplary embodiments, the side effects of treatment are reduced relative to a method that does not include step (i).

In a second aspect, the present invention is a method of treating non-melanoma skin cancers of the face in a subject in need thereof, comprising (i) applying heat to an affected area of the subject's skin using a thermal delivery device to achieve a skin temperature of between about 38 and about 42° C. for a suitable time; (ii) incubating a pharmaceutical composition comprising a photoactive agent for a period of less than about 14 hours to provide an incubated pharmaceutical composition; (iii) applying a therapeutically effective amount of the incubated pharmaceutical composition to the affected area; (iv) administering a suitable dose of red light to the affected area, therapy treating the non-melanoma skin cancer of the face.

In a particular embodiment, the non-melanoma skin cancer is basal cell carcinoma (BCC). The BCC may be primary, recurrent or incompletely excised previously.

In another particular embodiment, the non-melanoma skin cancer is a squamous cell carcinoma (SCC). The SCC may be primary, recurrent or incompletely excised previously.

In one embodiment, the non-melanoma skin cancer is a basal cell carcinoma and heat is applied in (i) for about thirty minutes.

In another embodiment, the non-melanoma skin cancer is basal cell carcinoma and heat is applied in (i) for about twenty minutes.

In exemplary embodiments, the side effects of treatment are reduced relative to method that do not include step (i).

In a particular embodiment, the incubation period is less than about 14 hours but achieves the same effect as if the pharmaceutical composition was incubated for about 14 hours in the absence of step (i).

In a particular embodiment, the incubation period is less than about 3 hours but achieves the same effect as if the pharmaceutical composition was incubated for about 3 hours in the absence of step (i).

In a particular embodiment, the incubation period is less than about 1 hour but achieves the same effect as if the pharmaceutical composition was incubated for about 1 hour in the absence of step (i).

In an exemplary embodiment, the incubation period is less than about 10, less than about 5 or less than about 1 minute.

In a third aspect, the present invention is a method of treating actinic keratosis (AK) of the face in a subject in need thereof, comprising (i) applying heat to an affected area of the subject's skin using a thermal delivery device to achieve a skin temperature of between about 38 and about 42° C. for a suitable time; (ii) incubating a pharmaceutical composition comprising a photoactive agent for a period of less than about 14 hours to provide an incubated pharmaceutical composition; (iii) applying a therapeutically effective amount of the incubated pharmaceutical composition to the affected area; (iv) administering a suitable dose of red light to the affected area, therapy treating the actinic keratosis.

In one embodiment, the affected area is heated for about 30 minutes.

In a particular embodiment, the affected area is heated to about 40° C.

In exemplary embodiments, the side effects of treatment are reduced relative to method that do not include step (i).

In a particular embodiment, the incubation period is less than about 14 hours but achieves the same effect as if the pharmaceutical composition was incubated for about 14 hours in the absence of step (i).

In a particular embodiment, the incubation period is less than about 3 hours but achieves the same effect as if the pharmaceutical composition was incubated for about 3 hours in the absence of step (i).

In a particular embodiment, the incubation period is less than about 1 hour but achieves the same effect as if the pharmaceutical composition was incubated for about 1 hour in the absence of step (i).

In an exemplary embodiment, the incubation period is less than about 10, less than about 5 or less than about 1 minute.

In a fourth aspect, the present invention is a method of treating disseminated superficial actinic porokeratosis (DSAP) of the face in a subject in need thereof, comprising) applying heat to an affected area of the subject's skin using a thermal delivery device to achieve a skin temperature of between about 38 and about 42° C. for a suitable time; (ii) incubating a pharmaceutical composition comprising a photoactive agent for a period of less than about 14 hours to provide an incubated pharmaceutical composition; (iii) applying a therapeutically effective amount of the incubated pharmaceutical composition to the affected area; (iii) administering a suitable dose of red light to the affected area, therapy treating the DSAP.

In exemplary embodiments, the side effects of treatment are reduced relative to method that do not include step (i).

In a particular embodiment, the incubation period is less than about 14 hours but achieves the same effect as if the pharmaceutical composition was incubated for about 14 hours in the absence of step (i).

In a particular embodiment, the incubation period is less than about 3 hours but achieves the same effect as if the pharmaceutical composition was incubated for about 3 hours in the absence of step (i).

In a particular embodiment, the incubation period is less than about 1 hour but achieves the same effect as if the pharmaceutical composition was incubated for about 1 hour in the absence of step (i).

In an exemplary embodiment, the incubation period is less than about 10, less than about 5 or less than about 1 minute.

### BRIEF DESCRIPTION OF THE FIGURES

FIG. 1: shows the results of treating inflammatory and cystic acne with the method of the present invention described in Example 1. As indicated in the figure, the subject remains clear for three months (FIG. 1B) and at least six months.

FIG. 2: shows the results of treating inflammatory and cystic acne with the method of the present invention a described in Example 1 at different time intervals and using a porphyrin camera (FIG. 2B).

FIG. 3: shows the results of treating nodular basal cell carcinoma on the extremities according to the method of the present invention described in Example 2. A wide margin of porphyrins is shown in the heated area in FIG. 3B using a porphyrin camera.

FIG. 4: shows the results of recurrent nodular and infiltrative basal cell carcinoma on the extremities according to the method of the present invention described in Example 3.

FIG. 5: shows the results of treating infiltrating basal cell carcinoma of the skull according to the method of the present invention described in Example 4.

FIG. 6: shows the results of treating infiltrating basal cell carcinoma of the skull according to the method of the present invention described in Example 5, including histology at baseline (FIG. 6A) and one (1) month post treatment (FIG. 6B), showing no residual BCC.

FIG. 7: shows the results of treating SCC-IS according to the method of the present invention described in Example 5. Heat is shown to create an increased porphyrin margin around the affected area, reflecting an increase in absorption.

FIG. 8: shows the results of treating multi-focal basal cell carcinoma using the method of the present invention.

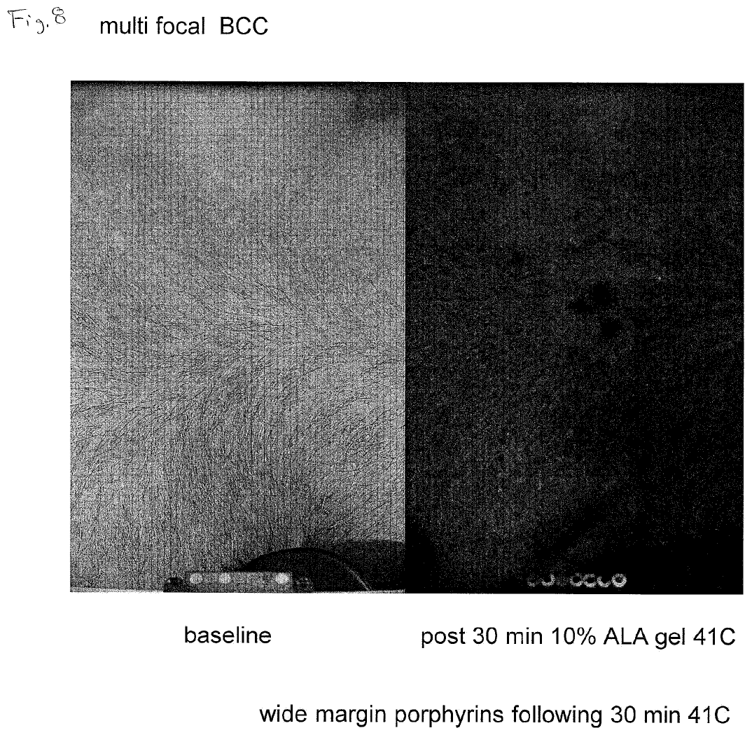

FIG. 9: shows the results of treating refractory disseminated porokeratosis with related SCC using the method of the present invention described in Example 6.

FIG. 10: shows the results of treatment as described in Example 7, including significant production of porphyrins after 20 minutes of incubation of the 20% ALA gel.

FIG. 11: shows the results of treatment as described in Example 8, including even greater production of porphyrins after 30 minutes of incubation of the 20% ALA gel.

FIG. 12: shows the results of treatment as described in Example 9, in the absence of heat, showing minimal production of porphyrins after 60 minutes incubation of the 20% ALA gel.

FIG. 13: shows porphyrin images during simultaneous incubation of a 20% ALA solution.

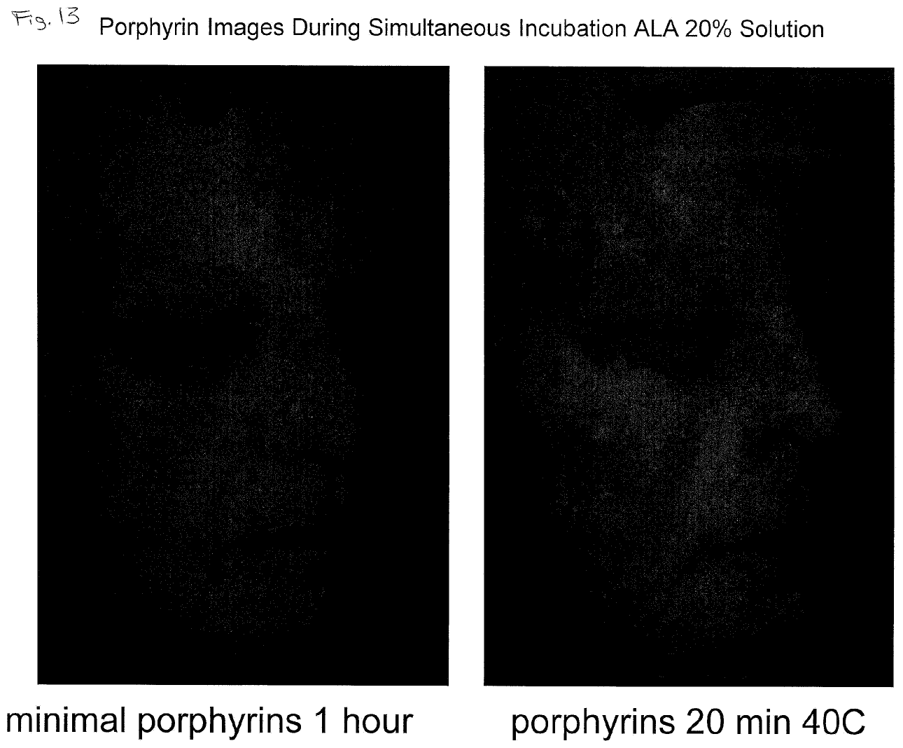

FIG. 14: shows images of treatment without heat (FIG. 14A) or with heat (FIG. 14B) using a 20% ALA solution, evidenced increased PDT reaction immediately post PDT on the heated side (FIG. 14C).

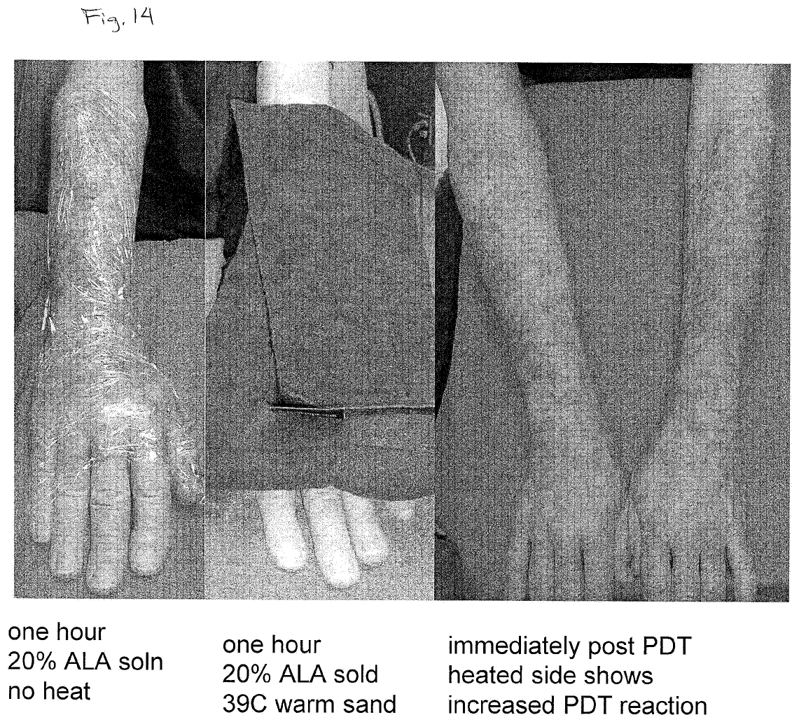

FIG. 15: shows the various settings utilized for the heating pad at one (1) minute and when measured between about 15 and about 60 minutes.

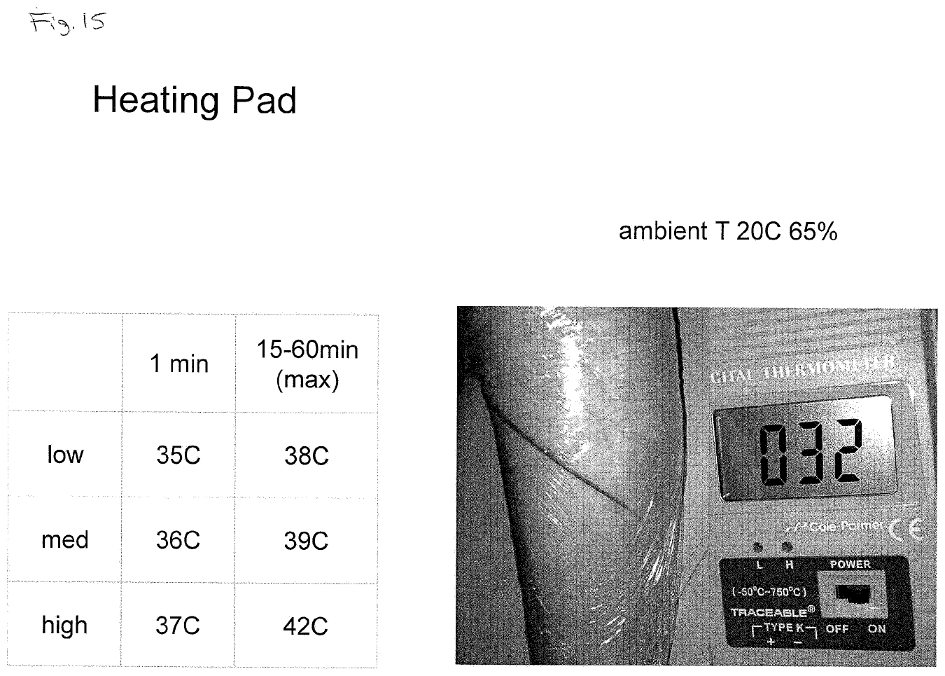

FIG. 16: shows the results of the method of the present invention employing a seventeen (17) minute incubation of a 20% ALA solution and utilizing a space heater with a temperature of between about 38° C. and about 42° C.

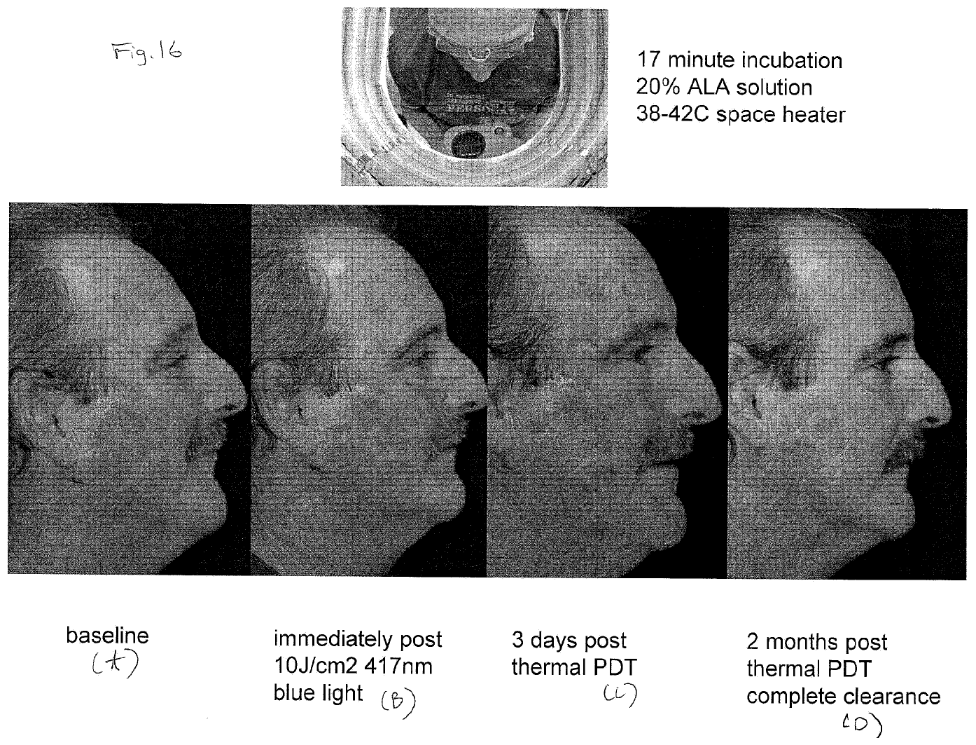

FIG. 17: shows the results of the method of the present invention employing a one hour incubation with 10% aminolevulinic acid (ALA) with a sodium acetate warming mask (skin temperature 40 C) followed by 37 J/cm2 635 nm red light when treating an index patient with moderate inflammatory and pustular acne on the face. The reduction in lesion counts was persistent 9 months after a single thermal PDT, as detailed in Example 10.

FIG. 18: shows the results of the method of the present invention employing a one hour incubation with 10% aminolevulinic acid with a sodium acetate warming mask (skin temperature 40 C) followed by 37 J/cm2 635 nm red light when treating an index patient with moderate inflammatory and pustular acne on the face. Remodeling of acne scars is shown 9 months after single thermal PDT, as detailed in Example 11.

FIG. 19: shows the margin of porphyrins created around a basal cell carcinoma (BCC) on the forearm greater than ten times the size of the original lesion following one hour incubation with ALA 20% under a heating pad set at medium (skin temperature 39° C.). The area of the BCC is 3,580² and the area of the BCC and porphyrins is 36,082².

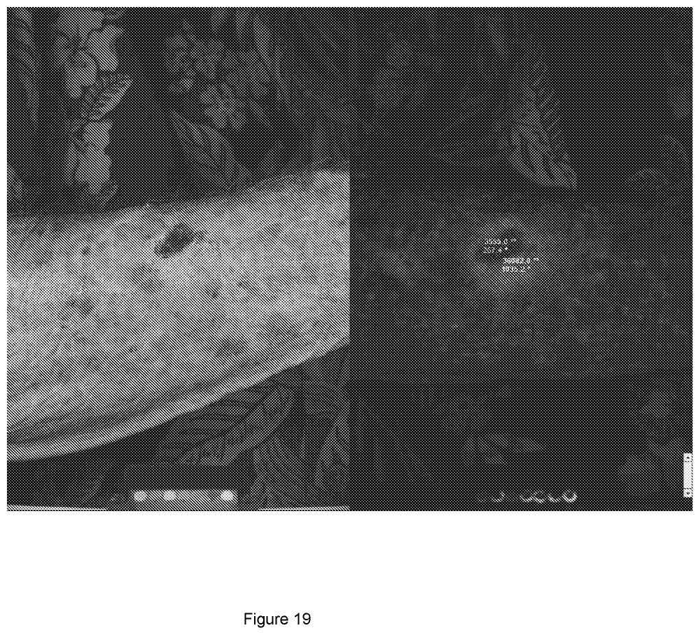

FIG. 20: shows the margin of porphyrins created around a squamous cell carcinoma (SCC) on the foot nine times the size of the original lesion following 30 minute incubation with ALA 20% under a sodium acetate warming pouch (skin temperature 42° C.). The area of the SCC is 1,051² and the area of the SCC and porphyrins is 9,269².

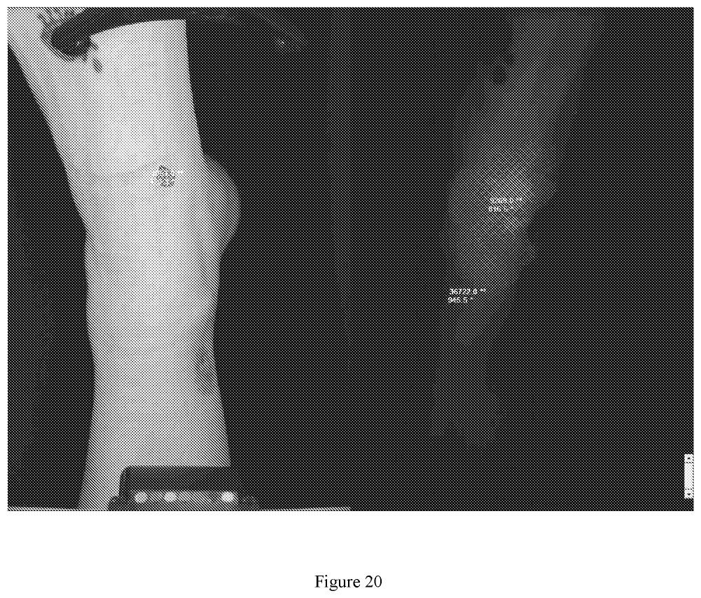

FIG. 21: shows clearance of basal cell carcinoma (BCC) on the chest 2 months following 30 minute incubation with ALA 10% under a sodium acetate warming pouch (skin temperature 42° C.) with margin of porphyrins demonstrated with porphyrin imaging.

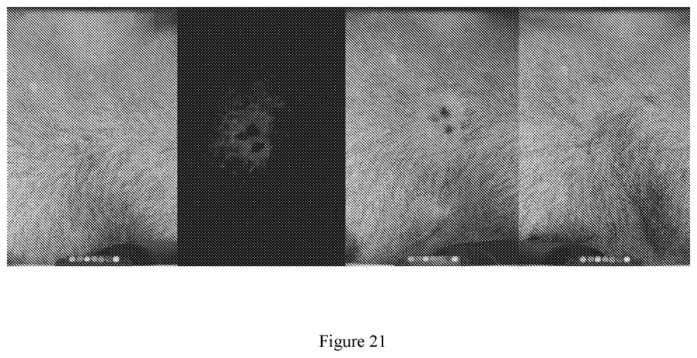

FIG. 22: shows the results of a split-face study with porphyrin imaging demonstrating the increase in porphyrin production on facial skin following incubation with 10% aminolevulinic acid (ALA) for 30 minutes with skin temperature at 40° C. (using a sodium acetate warming mask) compared to standard 3 hour incubation at room temperature (skin temperature at 30° C.).

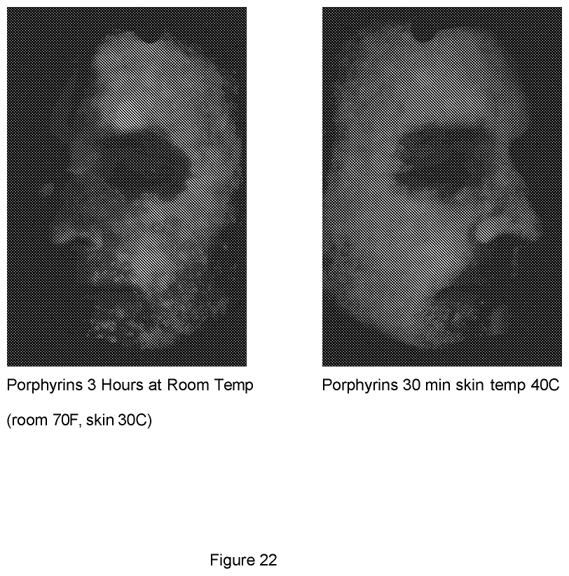

FIG. 23: shows the increased porphyrin production on facial skin with actinic keratoses following 30 minute incubation with 20% ALA at 40 C using a sodium acetate warming mask compared to the standard one hour incubation at room temperature (room temperature 70° F., skin temperature 30° C.). Increased porphyrin production was determined and shown as described in Example 12.

### DETAILED DESCRIPTION OF THE INVENTION

The present invention is based on the observation that heating of the skin increases the efficacy of photodynamic therapy (PDT), permitting a reduction in incubation time for the photosensitive agent (or pharmaceutical composition comprising the same) as well as increased absorption of the photoactive agent (or pharmaceutical composition comprising the same). In exemplary embodiments, the method of the present invention permits improved results, including complete clearance and/or reduced recurrence of the disease or disorder.

### I. Definitions

The term “acne” as used herein refers to inflammatory diseases of the pilosebaceous follicles and/or skin glands, and commonly is characterized by papules, pustules, cysts, nodules, comedones, other blemishes or skin lesions.

The term “actinic keratosis” or “AK” as used herein refers to precancerous skin lesions caused by and associated with chronic exposure to radiant energy, such as sunlight. Actinic keratosis lesions are small, red, rough hyperkeratotic lesions occurring on sun exposed areas of the skin.

The term ““administer” and “administration” as used herein refers to providing or causing the provision of a material to a subject, such as by a topical, subdermal, subcutaneous, intradermal, enteral, parenteral, rectal, nasal, intravenous, intramuscularly, intraperitoneal, or other route.

The term “basal cell carcinoma” or “BCC” as used herein refers to a malignant neoplasm of the keratinocytes that reside in the basal layer of the epidermis. It commonly develops on areas exposed to UV rays, particularly the head, face and neck. Although BCC does not typically spread beyond the primary site, it can be locally destructive. BCC may be classified as nodular ulcerative, superficial, pigmented or morpheaform.

The term “blue light” as used herein refers to light having a wavelength between about 380 nm and 500 nm, with visible blue light having a wavelength of about 475 nm.

The term “cancer” as used herein refers to a disease or disorder characterized by uncontrolled cell division and in many instances the ability of these cells to invade other tissues by the direct growth in adjacent tissue through invasion or by the implantation in distant sites through metastasis.

The term “drug-to-light interval” as used herein refers to the period of time between the administration of photosensitizing agent to the affected area and the administration of light to the affected area.

The term “duration of exposure” as used herein refers to the amount of time that skin is continuously exposed to a therapeutic agent (e.g., drug) or other therapeutic modality (e.g., heat, light).

The term “duration of treatment” as used herein refers to the amount of time between the initiation and completion of one cycle of treatment.

The term “effective amount” as used herein refers to an amount that elicits the biological or medicinal response in a tissue, system, animal, or human that is being sought, either alone or as a component of a therapeutic regime.

The term ““essentially no” as used herein in the context of tolerability refers to insignificant or de minimis occurrences of skin irritation events manifested in symptoms such as burning or stinging, flushing or blushing, or mild transient events.

The term “essentially no” as used herein in the contest of safety refers to insignificant or de Minimis occurrences of systemic or serious adverse events.

The term “heat-to-drug interval” as used herein refers to the period of time between the administration of heat to the affected area and the administration of the photosensitizing agent to the affected area.

The terms “high rates of clinical response” or “high efficacy” or “substantial decrease” in the context herein can relate to a reduction of about 45% or more in lesion count or to where subjects met a success criterion of “clear” or “almost clear” or to an “improvement of 2 grades from the baseline”.

The term “high risk location” as used herein with reference to a non-melanoma skin cancer refers to areas of the face including the central face, eyelids, eyebrows, periorbital, nose, lips [cutaneous and vermilion], chin, mandible, preauricular and postauricular skin/sulci, temple, ear); genitalia; hands, and feet.

The term “inhibiting” (and similar terms such as or “reducing” “lessening” or “preventing” or “avoiding” or any variation of these terms) as used herein refers to any measurable decrease or complete inhibition to achieve a desired result.

The term “lesion” as used herein refers to a diseased area of skin.

The term “lesion count” as used herein refers to the number of lesions (e.g., papules and pustules) present in a designated area of the body (e.g., in case of face, on the forehead, left and right cheeks, nose and chin).

The term “light” or “radiation” or similar terms as used herein includes all wavelengths. Preferably, the radiation wavelength is selected to match the wavelength(s) which excite(s) the photosensitizing agent. Even more preferably, the radiation wavelength matches the excitation wavelength of the photosensitive compound and has low absorption by the non-target tissues. The radiation is further defined in this invention by its intensity, duration, and timing with respect to dosing with the photosensitive agent. The intensity must be sufficient for the radiation to penetrate skin and/or to reach the target tissues to be treated. The duration must be sufficient to photoactivate enough photosensitive agent to act on the target tissues.

The term “low dose light” refers to a dosage of light, at a wavelength capable of being absorbed by the photosensitizer, that does not cause evident cell damage, necrosis, erythema or inflammation.

The term “low risk location” as used herein with reference to a non-melanoma skin cancer refers to areas of the trunk or extremities.

The term “margin” as used herein refers to the area immediately adjacent to, or surrounding, a tumor site but not showing any visible signs of carcinoma. The recommended “standard” surgical margins for various malignant lesions of skin are different.

The term “moderate risk location” as used herein with reference to non-melanoma skin cancer includes the cheeks, forehead, scalp, and neck.

The term “photodynamic therapy” or “PDT” as used herein refers to a technique involving the administration of administration of photosensitizing agents (e.g., to the skin or mucosa) followed by exposure to photoactivating light in order to activate the photosensitizing agents and convert them into cytotoxic form resulting in the destruction of cells and thus treatment of the disease. (See Kennedy J C. et al. J Photochem Photobiol B. 1990).

The terms “photosensitizing agent” or “photosensitizer” “or photoactive” are the like are used herein refers to a material, element, chemical, solution, compound, matter, or substance which is sensitive, reactive, receptive, or responsive to light energy. In certain embodiments, the photosensitizing agent may be activatable, i.e., controlled single 02 production that responds specifically to certain biomarkers.

The term “porphyrin” as used herein refers to a group of heterocyclic macrocycle organic compounds, composed of four modified pyrrole subunits interconnected at their a carbon atoms via methine bridges (═CH—). Expanded porphyrins result from the expansion of the phi electron conjugation by increasing the number of heterocyclic rings or bridging carbons of the existing porphyrin framework.

The term “red light” as used herein refers to light having a wavelength of between about 620 nm and about 750 nm.

The term “safe” as used herein means having no or essentially no adverse events (e.g., any unfavorable or unintended sign, symptom, or disease that appears or worsens on the course of treatment).

The term “scar” as used herein refers to marks created during the healing of damage to the skin or tissues. A scar is permanent and cannot be completely removed. Scars treated according to the method described herein include, for example, atrophic scar and hypertrophic scar. More particularly, the acne scar may be an ice pick, boxcar, rolling, bridges and tunnels, gross atrophy, dystrophic or keloid scars.

The term “target” as used herein refers to cells or tissue of the subject that is intended to be impaired or destroyed by the disclosed method. The target takes up the photosensitizing agent; then when sufficient radiation is applied, the target tissue is impaired or destroyed. Conversely, the term “non-target” as used herein refers to cells or tissue of the subject which are not intended to be impaired or destroyed by the treatment method. These non-target cells include but are not limited to those of other healthy tissues.

The term “therapeutically effective amount” as used herein refers to the amount of a therapeutic agent or modality that is sufficient to treat a subject. Effective amounts of the therapeutic agent and/or modality will vary according to factors such as the degree of susceptibility of the individual, the age, gender, and weight of the individual and idiosyncratic responses of the individual.

The term “topical” as used herein refers to directly laying on or spreading on the skin in need of the treatment, e.g., by use of the hands or an applicator.

The term “skin” as used herein refers to the multilayer organ including the epidermis, dermis, and hypodelinis (i.e., subcutaneous tissue) that covers the majority of the surface of mammalian subjects such as human beings, and also includes mucous membranes that are contiguous with the outer skin.

The term “skin cancer” as used herein refers to lentigo, maligna, melanoma, keratoacanthoma, basal cell carcinoma (BCC), squamous cell carcinoma (SCC), Merkel cell carcinoma (MCC), sarcoma, angiosarcoma, cutaneous lymphoma, sweat gland carcinoma and sebaceous gland carcinoma.

The term “squamous cell carcinoma” or “SCC” as used herein refers to a form of skin cancer that develops in the squamous cells of the skin, sometimes referred to as cutaneous squamous cell carcinoma. Similar to BCC, SCC tends to affect areas of skin with high levels of UV exposure such as the head, face and neck. SCCs are usually fast-growing and prone to metastasize. It may arise de novo or from actinic keratosis (approximately 0.5 to 16% conversion rate). It is generally classified as SCC in situ (involving the full thickness of the epidermis) or invasive (penetrating the basal membrane). Invasive SCC can be further classified as well, moderately or poorly differentiated.

The term “subject” as used herein refers to mammals, e.g., humans, companion animals (e.g., dogs, cats, birds, and the like), farm animals (e.g., cows, sheep, pigs, horses, fowl, and the like) and laboratory animals (e.g., rats, mice, guinea pigs, birds, and the like). In some embodiments, the subject is human.

The term “thermal treatment device” as used herein refers to any device that is capable of heating the skin, whether directly or indirectly.

The term “treat” or “treating” as used herein refers to (i) preventing or delaying the appearance of clinical symptoms of the disease developing in a mammal; (ii) inhibiting the disease i.e. arresting, reducing or delaying the development of the disease or a relapse thereof or

at least one clinical or subclinical symptom thereof, or (iii) relieving or attenuating one or more of the clinical or subclinical symptoms of the disease.

The term “topical” as used herein relates to the administration of a compound by means of the application on the body surface and includes but is not limited to transdermal administration and administration through the mucous membrane.

### II. Methods of Treatment

The present invention provides methods of treating disease or disorder of the skin using photodynamic therapy (PDT) in combination with heat.

Advantageously, the method of the present invention permits a reduction in incubation time for the photosensitive agent and/or duration of treatment compared to methods known in the art while achieving equivalent or superior results. In exemplary embodiments, the method of the present invention provides improved results relative to conventional treatments. When used to treat skin cancer, it permits an increase in complete incision rates resulting a reduction in recurrence or time to recurrence.

In one embodiment, present invention is a method of treating a skin disease or disorder in a subject in need thereof, comprising (i) administering heat to an affected area of the subject's skin using a thermal delivery device to achieve suitable temperature for a suitable time; (ii) incubating a pharmaceutical composition comprising a photoactive agent for a period of less than about 14 hours to provide an incubated pharmaceutical composition; (iii) administering a therapeutically effective dose of the incubated pharmaceutical composition to the affected area for a suitable time period; (iii) administering a dose of light to the affected area, thereby treating the skin disease or disorder.

Skin Diseases and Disorders

The disease or disorder of the skin treated by the method of the present invention may vary. In a particular embodiment, the skin disease or disorder is a cancerous legion, pre-cancerous lesion or acne. Areas of skin that can be treated include portions of skin on the head, face, neck, arms, legs, and torso, hands, and feet. In a particular embodiment, the area to be treated is the head face, neck or portions thereof.

Actinic Keratosis.

In one embodiment, the skin disorder treated according to the method of the present invention is a pre-cancerous lesion. In a particular embodiment, the pre-cancerous lesion is actinic keratosis (AK). AK (also known as solar keratosis) is the most common precancerous skin lesion, appearing as a crusty, scaly growth that often occurs in multiples. Actinic keratosis lesions generally measure in size between about 2 to about 7 millimeters in diameter. AK lesions can range in color from skin-toned to reddish and is often hyperkeratotic.

AK is typically caused by overexposure to ultra violet (UV) light. In rare instances, AK may be caused by overexposure to x-rays. These lesions typically appear on sun-exposed areas including the face, head (bald scalp), ears, shoulders, neck, arms, forearms and hands. In a particular embodiment, the disease or disorder treated according to the method of the present invention is AK on the face, neck or a portion thereof.

In one embodiment, the method of the present invention is used to treat a pre-cancerous lesion other than actinic keratosis. In a particular embodiment, the method of the present invention is used to treat a disease or disorder of the skin other than actinic keratosis.

Non-Melanoma Skin Cancer.

In another embodiment, the skin disorder treated according to method of the present invention is a cancerous lesion. In a particular embodiment, the cancerous lesion is a non-melanoma skin cancer (NMSC). Non-melanoma skin cancer includes all the types of cancer that occur in the skin that are not melanoma. The most common types of NMSC are basal cell carcinoma (BCC) and squamous cell carcinoma (SCC). BCC and SCC are also sometimes referred to as “keratinocyte carcinomas” to distinguish them from melanoma.

Basal cell carcinoma arises from basal keratinocytes of the epidermis, hair follicles, and eccrine sweat ducts. About 80% of skin cancers develop from this type of cell, typically on the head and neck (about 90%). BCC usually grows slowly (e.g., 1-2 cm over several years) and rarely spreads to other parts of the body (0.0003-0.05%). The tumor infiltrates tissues in a three-dimensional fashion through the irregular growth of subclinical finger-like outgrowths which remain contiguous with the main tumor mass. The actively growing tissue is at the periphery of the lesion, with cellular apoptosis and resultant ulceration in the central region.

The clinical features of BCC depend upon the subtype. Nodular BCC (also known as “classic basal-cell carcinoma”) is the commonest subtype (greater than 60% of cases), and presents as pink nodules, with rolled edges, surface telangiectasia and ulceration or crusting. Superficial BCCs account for up to 20% of cases and are found often on the trunk over sun-protected sites Appearing as pink scaly macules or thin plaques, they may be mistaken for Bowen's disease, psoriasis, discoid eczema or tinea corporis. Pigmented BCC occurs more commonly in patients from the Far East and may be mistaken for nodular melanoma. Morphoeic (sclerosing) BCC (also known as Cicatricial basal-cell carcinoma) has the worst prognosis and appears as subtle scar-like plaques, with ill-defined margin.

Histologically, BCC may be undifferentiated or differentiated. Undifferentiated BCC typically includes pigmented BCC, superficial BCC, sclerosing BCC and infiltrative BCC (a histologic subtype). Nodular BCC is typically differentiated. Other forms of differentiated BCC include keratotic BCC, BCC with sebaceous differentiation and adenoid BCC. Types of biopsy that may be used to confirm the diagnosis and determine the histologic subtype of BCC include shave biopsy and punch biopsy.

In one embodiment, the method of the present invention is used to treat undifferentiated BCC, and more particularly, superficial BCC.

In another embodiment, the method of the present invention is used to treat differentiated BCC, and more particularly, nodular BCC.

In exemplary embodiments, the BCC treated according to the method of the present invention has a mixed histological pattern, i.e., contains two or more major histologic patterns.

BCC is mainly caused by cumulative exposure to UV light, but can occur in subjects who have previously received radiation. Older age and male gender are associated with increased risk. Other risk factors include burn scars, small pox scars, xeroderma pigmentosa and basal cell nevus syndrome. Following development of a BCC, patients are at significantly increased risk of developing subsequent BCCs at other site.

In one embodiment, the method of the present invention may be used to treat BCC. In a particular embodiment, the method may be used to treat BCC selected from the group consisting of nodular, micronodular, cystic, infiltrative, superficial, pigmented, Rodent ulcer (also known as a “Jacobi ulcer”), fibroepithelioma of Pinkus, polyploid, pore-like or aberrant BCC.

In exemplary embodiments, the BCC treated according to the present invention is present on the head, neck or face. In one embodiment, the BCC is present at a target site selected from the nose, check, periorbital area, auricular area, ear, temple, forehead or scalp.

In exemplary embodiments, the BCC treated according to the present invention is present on the trunk, extremities, hands or feet.

The size of the lesion may vary. In one embodiment, the BCC is less than about 2 cm. In another embodiment, the BCC is greater than about 2 cm, greater than about 3 cm, greater than about 4 cm, greater than about 5 cm or greater than about 6 cm. Lateral delimitation of the lesion may be aided by dermoscopy and deep demarcation can be estimated by biopsy and/or imaging techniques.

The BCC may be primary, incompletely excised or recurrent. In a particular embodiment, the BCC is not primary. The BCC may be nodular or infiltrating in exemplary embodiments.

The BCC may be, with respect to recurrence, a low-risk, moderate-risk or high-risk BCC.

In one embodiment, the BCC treated according to the present invention is a low-risk BCC in terms of recurrence, based on location or size or both. In a particular embodiment, the lesion is in a low-risk location and <20 mm in size; in a moderate-risk location and <10 mm in size; or in a high-risk location and <6 mm in size. The lesion may be also be considered low-risk in view of the borders, i.e., well-defined.

In another embodiment, the BCC treated according to the present invention is a high risk (or higher risk) BCC in terms of recurrence, based on location or size or both. In a particular embodiment, the lesion is in a low-risk location and ≥20 mm in size; in a moderate-risk location and ≥10 mm in size; or in a high-risk location and ≥6 mm in size. The lesion may also be considered high-risk in view of the borders, i.e., poorly-defined, or because it is recurrent.

About 20% of skin cancers develop from squamous cells-flat, scale-like cells that make up most of the epidermis. SCC is more likely to spread to other parts of the body than BCC, although far less than melanoma.

The most common risk factor, like BCC, is cumulative exposure to UV radiation. Other risk factors include light coloring (e.g., blue eyes, blonde hair), chemical carcinogenesis, chronic radiation dermatitis, HPV, xeroderma pigmentosa and oculocutaneous albinism.

SCC may arise de novo or be proceeded by actinic keratosis (AK). There is a known conversion rate of about 0.0003 to about 0.05%. Histologically, AK is distinguished from SCC because the former involves only part of the epidermis. SCC in situ involves the full thickness of the epidermis and is well-defined and characterized by erythematous, scaly papules or plaques. Invasive SCC further involves penetration of the basement membrane of the epidermis and is characterized by indurated, scaling papules or plaques. Invasive SCC may be classified as well, moderately or poorly differentiated—where degree of differentiation is correlated with aggressiveness.

In one embodiment, the method of the present invention may be used to treat SCC. In a particular embodiment, the SCC is preceded by AK.

In another particular embodiment, the SCC is de novo, i.e., the patient has not been previously diagnosed with AK.

In one embodiment, the method of the present invention is used to treat SCC in situ.

In another embodiment, the method of the present invention is used to treat invasive SCC. In a particular embodiment, the method is used to treat invasive SCC selected from the group comprising well differentiated SCC, moderately differentiated SCC and poorly differentiated SCC.

The location of the lesion may vary. In exemplary embodiments, the SCC treated according to the present invention is present on the head, neck or face. In one embodiment, the SCC is present at a target site selected from the nose, check, periorbital area, auricular area, ear, temple, forehead or scalp.

The size of the lesion may vary. In one embodiment, the size of the SCC is less than about 2 cm. In another embodiment, the size of the SCC is greater than about 2 cm, greater than about 3 cm, greater than about 4 cm, greater than about 5 cm or greater than about 6 cm.

The thickness of the lesion may vary. In one embodiment, the thickness of SCC is less than about 2 mm. In another embodiment, the thickness of the SCC is greater than about 2 mm, greater than about 4 mm, greater than about 6 mm or greater than about 6 mm. In exemplary embodiments, the SCC is beyond subcutaneous fat or characterized by perineural invasion (PNI).

The SCC may be primary, incompletely excised or recurrent. In a particular embodiment, the SCC is not primary. In exemplary embodiments, the SCC is loco-regionally recurrent (LRR).

The SCC may be, with respect to recurrence, a low-risk, moderate-risk or high-risk SCC.

In one embodiment, the SCC treated according to the present invention is a low-risk SCC in terms of recurrence, based on location or size or both. In a particular embodiment, the lesion is in a low-risk location and <20 mm in size; in a moderate-risk location and <10 mm in size; or in a high-risk location and <6 mm in size. The lesion may be also be considered low-risk in view of the borders, i.e., well-defined, or because it is well-differentiated or has a depth of <2 mm.

In another embodiment, the SCC treated according to the present invention is a high risk (or higher risk) SCC in terms of recurrence, based on location or size or both. In a particular embodiment, the lesion is in a low-risk location and ≥20 mm in size; in a moderate-risk location and ≥10 mm in size; or in a high-risk location and ≥6 mm in size. The lesion may also be considered high-risk in view of the borders, i.e., poorly-defined, or because it is recurrent. The lesion may also be considered high risk because it is poorly differentiated or has a depth of >2 mm.

Other non-melanoma skin cancers that can be treated according to the methods of the present invention include including Merkel cell carcinoma, cutaneous (skin) lymphomas, Kaposi sarcoma, skin adnexal tumors, and sarcomas.

Acne.

In a further embodiment, the skin disease or disorder is acne. Acne is a common skin condition which can occur anywhere on the body, but often on the face and back. It typically occurs during puberty, but can occur at any age. Without treatment, dark spots and scars can appear on the skin as acne clears.

Acne may be caused by one factor, or a combination of factors, which generally include genetic predisposition, Excessive production of sebum by the sebaceous glands, alterations of the keratinization process with abnormal desquamation of the sebaceous follicular epithelium, proliferation of Propionibacterium acnes and release of skin inflammatory mediators.

Acne can be classified as non-inflammatory or inflammatory. Non-inflammatory acne generally includes whiteheads (typically small and remaining under the skin) and blackheads (typically visible).

Inflammatory acne includes papules (small, usually pink bumps), pustules (red at their base and have pus at the top), nobules (large, solid, painful pimples that are embedded deep in the skin) and cysts (painful and filled with pus). Factors contributing to inflammation include bacterial infection (Propionibacterium acnes), free fatty acids and sebum, pro-inflammatory mediators (IL-1a, IL-b, TNF) and debris.

Representative, non-limiting types of acne that can be treated by the methods disclosed herein include acne vulgaris, acne comedo, papular acne, premenstrual acne, preadolescent acne, acne venenata, acne cosmetica, pomade acne, acne detergicans, acne excoriee, gram negative acne, pseudofolliculitis barbae, folliculitis, perioral dermatitis, hiddradenitis suppurativa, cystic acne, acne atrophica, bromide acne, chlorine acne, acne conglobata, acne detergicans, epidemic acne, acne estivalis, acne fulminans, halogen acne, acne indurata, iodide acne, acne keloid, acne mechanica, acne papulosa, pomade acne, premenstral acne, acne pustulosa, acne scorbutica, acne scrofulosorum, acne urticata, acne varioliformis, acne venenata, propionic acne, acne excoriee, gram negative acne, steroid acne, nodulocystic acne and acne rosacea.

A grading system may also be used to categorize the various stages of the acne lesion. Grade one, the microcomedone, can appear as whiteheads or blackheads. Grade two is a papule, i.e., a small pink inflamed bump. Grade three is a pustule lesion with more visible inflammation than papule. Grade four is a nodule or large painful solid lesion that extends deep into the skin. Grade five is an inflamed lesion, very large and painful.

In one embodiment, the disease or disorder treated according to the method of the present invention is acne. In exemplary embodiment, the disease or disorder is inflammatory acne on the face, neck or portion thereof. The inflammatory acne may be mildly inflammatory, moderately inflammatory or severely inflammatory.

In one embodiment, the disease or disorder treated according to the method of the present invention is mild acne. Mild acne is generally categorized by the appearance of with blackheads and whiteheads, but can also include papules and pustules.

In another embodiment, the disease or disorder treated according to the method of the present invention is moderate acne. Moderate acne is generally characterized by appearance of more painful, deep-rooted, inflamed lesions, which can result in scarring

In a further embodiment, the disease or disorder treated according to the present invention is severe acne. Severe acne is generally characterized by the appearance of deep-rooted inflammatory lesions, including cysts and nodules which can be painful and can produce scarring.

In one embodiment, the disease or disorder treated according to the present invention is not severe acne.

In one embodiment, the subject treated for acne according to the present invention is an adolescent. In another embodiment, the subject is an adult.

Porokeratosis. In one embodiment, the method of the present invention is used to treat prokeratosis. Porokeratosis is a clonal disorder of keratinization characterized by one or more atrophic patches surrounded by a clinically and histologically distinctive hyperkeratotic ridgelike border called the cornoid lamella.

The method of the present invention may be used to treat any form of prokeratosis. Various forms are recognized including classic porokeratosis of Mibelli (PM); disseminated superficial porokeratosis (DSAP), disseminated superficial prokeratosis (DSP); linear porokeratosis, prokeratosis palmaris et plantaris disseminata (PPPD) and punctate porokeratosis, which might represent a variant of PPPD. Other less common forms are also recognized in the literature.

In exemplary embodiments, the method of the present invention is used to treat DSAP. In exemplary embodiments, the method of the present invention is used to treat PM.

In certain embodiments, the method of the present invention is used to treat multiple lesions simultaneously.

Heating

Heat by administered to the skin by any suitable thermal delivery device. The skin should be clean and dry. The device may be configured for direct application to the skin, or to provide heat indirectly. The energy source may be, for example, electrical, chemical, a laser, a microwave or radiofrequency. Representing, non-limiting thermal delivery devices include heating pads, heating masks, space heaters or infrared heaters.

In one embodiment, the affected area is the face and the thermal delivery device is a heat mask. In exemplary embodiments, the heat mask is a sodium acetate mask that heats upon crystallization. Crystallization is typically triggered by flexing a small flat disc of notched ferrous metal embedded in the super saturated sodium acetate liquid. In exemplary embodiments, the mask is covered with two layers of 2 play poly plastic paper.

In another embodiment, the affected area is the extremities and the thermal delivery device is a heating pad. The heating pad may be placed in any suitable manner.

In certain embodiments, a space heater is used to heat the facial skin, e.g., placing a space heater on a stand with wheels. The subject controls the skin heating at an ideal distance (adjusting for comfort) warming the skin. In this embodiment, the subject should wear protective eye wear (e.g., goggles).

In one embodiment, the skin is not heated by simply heating the room itself.

The temperature to which the thermal delivery device is heated may vary, depending on the disease or disorder being treated and the location. In one embodiment, the thermal delivery device is heated to a temperature of between about 20 to about 50° C., or more particularly, about 20 to about 30° C., about 30 to about 40° C., or about 40 to about 50° C.

In one embodiment, the disease or disorder is a pre-cancerous or cancerous lesion on the face and the thermal delivery device is heated to a temperature between about 38 and about 42° C., or more particularly, about 40° C.

In another embodiment, the disease or disorder is a pre-cancerous or cancerous lesion on the extremities and the thermal delivery device is heated to a temperature between about 38 and about 42° C., or more particularly, about 39° C.

In exemplary embodiments, the skin is heated to the temperature of the thermal delivery device. In exemplary embodiments, the temperature of the skin at the surface is less that the temperature to which the thermal delivery device is heated.

In one embodiment, the skin is heated to a surface temperature of between about 20 to about 50° C., or more particularly, about 20 to about 30° C., about 30 to about 40° C., or about 40 to about 50° C.

In another embodiment, the skin is heated to a surface temperature of greater than about 37° C. In a particular embodiment, the skin is heated to a surface temperature greater than about 38, greater than about 39, greater than about 40, greater than about 41 or greater than about 42° C.

In one embodiment, the skin is heated to a surface temperature between about 38 and about 42° C., or more particularly, about 40° C.

In another embodiment, skin is heated to a surface temperature between about 38 and about 42° C., or more particularly, about 39° C.

The duration of exposure to heat may vary. In one embodiment, the duration of exposure is between about 1 and about 90 minutes, or more particularly, between about 1 and about 10 minutes, about 10 and about 20 minutes, about 20 and about 30 minutes, about 30 and about 40 minutes, about 40 and about 50 minutes, about 60 and about 70 minutes, about 70 and about 80 minutes, or about 80 and about 90 minutes or more.

In another embodiment, the duration of exposure to heat is about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, or about 80 minutes or greater.

In a particular embodiment, the skin disease or disorder is a pre-cancerous lesion and the duration of exposure to heat is about between about 20 and about 40 minutes, or more particularly, about 30 minutes.

In yet another embodiment, the skin disease or disorder is acne and the duration of exposure to heat is between about 20 and about 40 minutes, or more particularly, about 30 minutes.

Without being bound by any particular theory, it is believed that heating increases the one or more of: The rate of porphyrin production in the skin, the absorption of the photosensitive agent or the area of treatment surrounding the lesion so as to create a margin of treatment around the lesion.

In exemplary embodiments, the rate of porphyrin production in the skin is increased by about 10, about 20, about 30, about 40 or about 50% or more by heating as disclosed herein.

In exemplary embodiments, absorption is increased by about 10, about 20, about 30, about 40 or about 50% or more by heating as disclosed herein. The absorption may reflect an increased ratio of photosensitive agent absorption: lesion depth or and increased ratio of photosensitive agent absorption to lesion with. In a particular embodiment, the ratio if between about 0.5:1 to about 1:1, more particularly, about 0.5:1, about 0.6:1, about 0.7:1, about 0.8:1, about 0.9:1 or about 1:1 or greater.

In exemplary embodiments, the area of treatment is increased by about 10, about 20, about 30, about 40 or about 50% or more by heating as disclosed herein.

Optionally, the method may comprise one or more additional pre-treatment steps, i.e., before application of the photosensitive agents. In one embodiment, the pre-treatment may comprise curettage, dermoabrasion (e.g. with sandpaper) or micro-perforation of the skin.

Photosensitizing Agents/Composition

The present invention involves application of a photosensitizing agent(s) to skin, which accumulates in the affected area to a much higher degree than they accumulate in surrounding normal tissues. When photosensitizing agents are irradiated with an suitable dose of light, the activated agents pass on their energy to surrounding molecular oxygen to produce reactive oxygen species (ROS) in cells that have retained the agent. When an appropriate dose of light is utilized, the result is cell death.

Photosensitizing agents can be administered orally or parenterally, neat or in combination with conventional pharmaceutical carriers.

Any suitable photosensitizing agent can be used in the method of the present invention. Numerous photosensitive agents are known in the art. In one embodiment, the photosensitizer is selected from the group consisting of porphyrins, chlorophylls, and dyes.

Non-limiting examples of photosensitive agents suitable for use in the present invention include phthalocyanines, porphyrins, porphyrin precursors, porphycenes, naphthalocyanines, phenoselenazinium, hypocrellins, perylenequinones, texaphyrins, benzoporphyrin derivatives, azaporphyrins, purpurins, Rose Bengal, xanthenes, porphycyanines, isomeric porphyrins, pentaphyrins, sapphyrins, chlorins, benzochlorins, hypericins, anthraquinones, rhodanols, barbituric acid derivatives, expanded porphyrins, dipyrromethenes, coumarins, azo dyes, acridines, rhodamine, azine derivatives, tetrazolium derivatives, safranines, indocyanines, indigo derivatives, indigo triazine derivatives, pyropheophorbides, pyrrole derived macrocyclic compounds, naturally occurring or synthetic porphyrins, naturally occurring or synthetic chlorins, naturally occurring or synthetic bacteriochlorins, naturally occurring or synthetic isobacteriochlorins, naphthalocyanines, phenoxazine derivatives, phenothiazine derivatives, chalcoorganapyryllium derivatives, triarylmethane derivatives, rhodamine derivatives, fluorescein derivatives, verdin derivatives, toluidine blue derivatives, methylene blue derivatives, methylene violet derivatives, nile blue derivatives, nile red derivatives, phenazine derivatives, pinacyanol derivatives, plasmocorinth derivatives and indigo derivatives, and combinations thereof.

In one embodiment, the photosensitizing agent is a porphyrin. In one embodiment, the porphyrin is selected from the group consisting of hematoporphyrins, metalloporphyrins, porphycenes, pheophorbides, purpurins, chlorins, protoporphyrins and phthalocyanines.

In a particular embodiment, the photosensitizing agent is 5-aminolevulinic acid (ALA) or a derivative or modification thereof. 5-aminolevulinic acid (also known as 5-aminolaevulinic

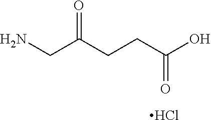

acid, delta-aminolevulinic acid, delta-aminolaevulinic acid, or 5-amino4-oxopentanoic acid) is an intermediate in the pathway to the production of the photosensitizer, proptoporphyrin IX (PpIX).

### 5-Aminolevulinic Acid HCL

ALA can be used in the form of a salt, such as a HCL, or in a pharmaceutically equivalent form, such as an amide or ester. As used herein the term “ALA” refers to all of the above-referenced compounds.

In one embodiment, the photosensitizing agent is a non-porphyrin agent. In a particularly embodiment, the non-porphyrin agent is selected from the group consisting of anthraquinone, phenothiazine, psoraleans, anthracyclines, chalcogenopyrlium dyes, xanthene, cyanine, and curcuminoid sensitizers.

The photosensitizing agent can be administered in pure form, applied after being dissolved in a non-toxic solvent, or it can be administered in a topical formulation.

When formulated as a pharmaceutical composition, the composition contains a pharmaceutically acceptable carrier and optionally, one or more of the following agents: buffers, co-solvents, adsorbents, permeation enhancers, surfactants, stabilizers, emulsifiers, preservatives, chelating agents, thickening agents, smoothing agents, humectants or polymers.

The amount of the photosensitizing agent in the pharmaceutical composition (e.g., topical dosage form) may vary. In one embodiment, the photosensitizing agent is present in an amount from about 0.1 wt. % to about 75 wt. %, and more particularly, about 1.0 wt. % to about 40 wt. %, about 2.0 wt. % to about 30 wt. %, about 5.0 wt. % to about 25 wt. %, about 10 wt. % to about 20 wt. %, about 8 wt. %, about 10 wt. %, about 12 wt. %, about 14 wt. %, about 16 wt. %, about 18 wt. %, about 20 wt. % or about 22 wt. %.

In a particular embodiment, the amount of the amount of the photosensitizing agent in the pharmaceutical composition is greater than about 10 wt. %.

In a particular embodiment, the amount of the photosensitizing agent in the pharmaceutical composition is about 20 wt. %.

In another embodiment, the amount of the photosensitizing agent in the pharmaceutical composition is less than about 20 wt. %, and more particularly, less than about 15 wt. % or about 10 wt. % but in each case greater than zero.

In one embodiment, the photosensitizing agent is formulated for topical delivery. Topical dosing forms can include creams, gels, ointments, pastes, suspensions, lotions, foams, sprays, aerosols, and solutions.

The term “cream” refers to semi-solid emulsion systems which contain both an oil and water. Oil in water creams are water miscible and are well absorbed into the skin, Aqueous Cream BP. Water in oil (oily) creams are immiscible with water and, therefore, more difficult to remove from the skin. These creams are emollients, lubricate and moisturize, e.g., Oily Cream BP. Both systems require the addition of either a natural or a synthetic surfactant or emulsifier.

The term “ointment” refers to systems which have oil or grease as their continuous phase. Ointments are semi-solid anhydrous substances and are occlusive, emollient and protective. Ointments restrict transepidermal water loss and are therefore hydrating and moisturizing. Ointments can be divided into two main groups-fatty, e.g., White soft paraffin (petrolatum, Vaseline), and water soluble, e.g., Macrogol (polyethylene glycol) Ointment BP.

The term “lotion” refers to those solutions typically used in dermatological applications.

The term “gel” refers to semi-solid permutations gelled with high molecular weight polymers, e.g., carboxypolymethylene (Carbomer BP) or methylcellulose, and can be regarded as semi-plastic aqueous lotions. They are typically non-greasy, water miscible, easy to apply and wash off, and are especially suitable for treating hairy parts of the body.

In one embodiment, the photosensitive agent is formulated as a gel. Pharmaceutically acceptable gelling agents suitable for use in the topical gel composition are those agents which provide viscosity to a formulation such that the formulation can be effectively applied to the affected area, including but not limited to hydroxypropylcellulose (e.g. Klucel H A), hydroxypropyl methylcellulose carrageenans, microcrystalline cellulose, carbomer, alginates, gellan gum, xanthan gum, veegum, hydroxyethyethyl cellulose, guar gum and carbomers

The gel can further include local anesthetics and analgesics, such as camphor, menthol, lidocaine, dibucaine, and pramoxine; antifungals, such as ciclopirox, chloroxylenol, triacetin, sulconazole, nystatin, undecylenic acid, tolnaftate, miconazole, clotrimazole, oxiconazole, griseofulvin, econazole, ketoconozole, and amphotericin B.

The gel can also include one or more antiseptics, such as iodine, povidine-iodine, benzalkonium chloride, benzoic acid, nitrofurazine, benzoyl peroxide, hydrogen peroxide, hexachlorophene, phenol, resorcinol, and cetylpyridinium chloride.

The pH of the gel formulations of the photosensitizing agents are preferably within a physiologically acceptable pH range, e.g., within the range of about 4.5 to about 7.5, more preferably, of about 5.0 to about 6.5, such as a pH of about 5.1, 5.15, 5.2, 5.25, 5.3, 5.35, 5.4, 5.45, 5.5, 5.55, 5.6, 5.65, 5.7, 5.75, 5.8, 5.85, 5.9, 5.95, 6.1, 6.15, 6.2, 6.25, 6.3, 6.35, 6.4, 6.45, or 6.5. To stabilize the pH, preferably, an effective amount of a buffer is included. Acids or bases can be used to adjust the pH as needed.

The gel composition can be prepared by mixing the ingredients of the composition according to known methods in the art, for example methods provided by standard reference texts such as: Remington: The Science and Practice of Pharmacy, 1577-1591, 1672-1673, 866-885 (Alfonso R. Gennaro ed., 19th ed., 1995); Ghosh, T. K. et al., Transdermal And Topical Drug Delivery Systems (1997), both of which are hereby incorporated herein by reference.

In an exemplary embodiment, the photosensitizing agent is formulated as a gel comprising 20% aminolevulinic acid hydrochloride gel. In a particular embodiment, the pharmaceutical composition is LEVULAN® KERASTICK®, a topical formulation of 20% 5-aminolevulinic acid hydrochloride. In another particular embodiment, the pharmaceutical composition is not LEVULAN® KERASTICK®.

In a further embodiment, the photosensitizing agent is formulated as a gel comprising 10% aminolevulinic acid hydrochloride. In a particular embodiment, the pharmaceutical composition is AMULEZ® (from Biofrontera), a non-sterile topical formulation of 10% 5-aminolevulinic acid hydrochloride (equaling 7.8% of free acid) in a gel-matrix with nanoemulsion.

In one embodiment, the the photosensitizing agent is in unit dosage for, such that the photosensitizing agent can be sub-divided in unit dose(s) containing appropriate quantities of the compound.

The quantity of photosynthesizing agent to be administered depends on the choice of agent, the condition to be treated, the mode of administration, the individual subject, and the judgment of the practitioner. Depending on the specificity of the preparation, smaller or larger doses may be needed. In one embodiment, one gram (78 mg) of 5-aminolaevulinic acid hydrochloride gel (ALA) is administered.

The time between application of heat to the affected area and application of the photosensitizing agent may vary. This is known as the “heat-to-drug interval” and may be measured in seconds, minutes, hours or even days.

In one embodiment, the heat-to-drug interval is about 1 to about 60 seconds, or more particularly, about 1 to about 10 seconds, about 10 to about 20 seconds, about 20 to about 30 seconds, about 30 to about 40 seconds, about 40 to about 50 seconds, or about 50 to about 60 seconds.

In another embodiment, the heat-to-drug interval is about 1 to about 60 minutes, and more particularly, about 0.1 to about 1 minute, about 1 minute to about 5 minutes, about 5 minutes to about 10 minutes, about 10 minutes to about 20 minutes, about 20 minutes to about 30 minutes, about 30 minutes to about 40 minutes, about 40 minutes to about 50 minutes, or about 50 minutes to about 60 minutes.

In further embodiment, embodiment, the heat-to-drug interval is about 1 and about 24 hours, more particularly, about 1 to about 4 hours, about 4 to about 8 hours, about 8 to 12 hours, about 12 to about 16 hours, about 16 to about 20 hours, or about 20 to about 24 hours.

The composition can be administered in a suitable manner, preferably topical. A topical composition may be administered in any suitable manner, e.g., rubbed on, poured on, applied with an applicator (e.g., a gauze pad, a swab, a bandage, etc.), or the like. In some cases, the composition can be a liquid, a gel, a cream, a lotion, an ointment, a solid “stick,” or the like, that can be applied to the skin by hand, for example, by rubbing or spraying.

In exemplary embodiments, the affected area is the site of a precancerous or cancerous lesion, for example, on the face or neck. In some embodiments, the affected area is an area that covers the site of the precancerous or cancerous lesion and is, for example, about 1, 2, 3, 4, 5, 6, 7, 8, 9, 10, 15, 20, 25, 30, 40, or 50 cm in diameter. In some embodiments, the suitable location is an area where cancer prevention is desired. In other embodiments, the suitable location is a site where a pre-cancerous or cancerous lesion has previously been removed and the method is designed to prevent reoccurrence. In yet another embodiment, the suitable location is a site where a pre-cancerous or cancerous lesion has previously been incompletely removed.

In exemplary embodiments, the affected area is the site of an acne lesion, for example, on the face. In some embodiments, the affected area is an area that covers the site of the acne and is, for example, about 1, 2, 3, 4, 5, 6, 7, 8, 9, 10, 15, 20, 25, 30, 40, or 50 cm in diameter. In some embodiments, the suitable location is an area where acne prevention is desired.

The duration of exposure to the photosensitive agent may vary. In one embodiment, the duration of exposure is between about 1 and about 30 minutes, or more particularly, about 1, about 5, about 10, about 15, about 20, about 25 or about 30 minutes.

In exemplary embodiments, the duration of exposure to the photosensitive agent may be less while achieving the same effect due to pre-heating of the skin. In one embodiment, the duration of exposure is about 10, about 30, about 30, about 40, about 50 or about 60%, about 70%, about 80%, or about 90% while achieving the same effect.

In a particular embodiment, the duration of exposure to the photosensitive agent is about 1 hour, about 45 minutes, about 30 minutes or less than about 30 minutes.

Incubation Time

One advantageous feature of the present invention is the reduction it permits in incubation time for the photosensitive agent. The FDA approval for PDT for AK requires a 14 hour incubation with ALA before exposure to the blue light. Yet, for many clinicians, that creates a burden including extended duration of treatment.

In one embodiment, the incubation time is reduced by greater than about 10%, greater than about 15%, greater than about 20%, greater than about 25%, greater than 30%, greater than about 35%, greater than 40%, greater than about 45%, greater than about 50%, greater than about 54%, greater than 60%, greater than about 65%, greater than 70%, greater than about 75% or greater than 80%.

In another embodiment, the incubation time is reduced by between about 10% and about 70%, about 20% and about 60%, about 30% and about 50%.

In a particular embodiment, the incubation time is reduced by greater than 30% while achieving equivalent results to a photoactive agent incubated for about 14 hours.

In another particular embodiment, the incubation time is reduced by greater than 20% while achieving equivalent results to a photoactive agent incubated for about 5 hours.

In another particular embodiment, the incubation time is reduced by greater than 10% while achieving equivalent results to a photoactive agent incubated for between about 1 and about 3 hours, or more particular, about 3 hours, about 2 hours or about 1 hour.

In another embodiment, the incubation time is less than 14 hours, less than 13 hours, less than 12 hours, less than 11 hours, less than 10 hours, less than 8 hours, less than 7 hours, less than 6 hours, less than 5 hours, less than 4 hours, less than 3 hours, less than 2 hours or less than 1 hour.

In a further embodiment, the incubation time is about 1 hour, about 2 hours, about 3 hours, about 4 hours, about 5 hours, about 6 hours, about 7 hours or about 8 hours or more.

In exemplary embodiments, the incubation time is less than about 1 minute, less than about 45 seconds, less than about 30 seconds, less than about 25 seconds, less than about 20 seconds, less than about 15 seconds or less than about 10 seconds.

In exemplary embodiments, the incubation time is about 30 seconds, about 25 seconds, about 20 seconds, about 18 seconds, about 16 seconds, about 14 seconds, about 12 seconds or about 10 seconds or less.

In exemplary embodiments, the photosensitizing agent is incubated simultaneously with application of heat to the skin or within a few seconds or minutes prior to heating of the skin or after heating of the skin has begun.

Light

After the skin has been heated and the photosensitizer applied, the area to be treated is then irradiated with light of a proper wavelength and sufficient power in the presence of oxygen to activate the photosensitive agent, resulting in reactive oxygen species. These reactive oxygen species react with biomolecules, fatally damaging some of the cells in the treatment area.

The step of irradiating generally comprises the step of providing a light source that is activated to produce the light. The light source may be an artificial or natural light source (e.g., sunlight).

The light can be provided by a laser, light emitting diode LED), or other light source known to those skilled in the art (e.g., chemiluminescent light source). The light can be provided by a single source, or a plurality of light sources can be used.

In an exemplary embodiment, the light source is an LED lamp.

The amount and wavelength of radiation applied is dependent on the nature of the photosensitizing agent used. Preferably, the wavelength is selected to correspond with or at least overlap the excitation wavelength(s) of the photosensitizing agent. Even more preferably, the wavelength matches the excitation wavelength of the photosensitizing agent and has low absorption by the non-target cells or tissues.

The wavelength is typically within the wavelengths of visible and near infra-red light, which includes wavelengths from about 320 nm to about 780 nm. However, specific photosensitizers typically respond to light having a particular wavelength.

In one embodiment, the light administered to the affected area is blue light. Blue light has a wavelength between about 380 nm and 500 nm, with visible blue light having a wavelength of about 475 nm. The dominant wavelength of the blue light used in the method of the present invention may vary. In one embodiment, it is between about 450 and about 475 nm. In a particular embodiment, it is about 450 nm, about 455 nm, about 460 nm, about 465 nm, about 470 nm or about 475 nm. The source of the blue light may vary. In one embodiment it is Blu-U® from DUSA Pharmaceuticals.

In one embodiment, the light is not blue light. In a particular embodiment, the light is not Blu-U®.

In another embodiment, the light is not blue light and the photosensitive agent is not LEVULON® KERASTICK®.

In another embodiment, the light is red light. Red light has a wavelength from about to about 620 to about 750 nm. The dominant wavelength of the red light used in the method of the present invention is between about 630 nm and about 640 nm, or more particularly about 635 nm. The source of the red light may vary. In one embodiment, it is BF-RhodoLED® (from BioFrontera).

In certain embodiments, the administration of light is continuous.

In exemplary embodiments, the light source is disposed external to an intact skin layer of the subject.

In other embodiment, more than one administration of light can be used within a single treatment, e.g., two, three, four or more discrete administrations. The amount of light applied in the two more administrations may be the same or different. Once the first application of light has been applied to the effective area, a sufficient interval of time should be allowed to pass for an effective amount of the photosensitizing agent to further penetrate the tissue. The particular layer of skin targeted can vary depending on the disease being treated.

The duration of exposure to light may vary depending on the power of the radiation source. In one embodiment, the duration of exposure is between about 1 to about 30 minutes, or more particularly about 1 to about 5 minutes, about 5 to about 10 minutes, about 10 to about 15 minutes, about 15 to about 20 minutes, about 20 to about 25 minutes or about 25 to about 30 minutes or longer.

In another embodiment, the duration of exposure is about 1 minute, about 3 minutes, about 5 minutes, about 8 minutes, 10 minutes, about 12 minutes, about 15 minutes, about 18 minutes, or about 21 minutes, about 25 minutes, about 28 minutes, or about 31 minutes or more.

The light should be administered for a period of time and intensity sufficient to activate the photosensitizing agent. The amount of light energy necessary to obtain effective results can be pre-determined by evaluating the effective amounts for particular subjects, types of subjects, photosensitizers, and/or formulations. The amount of light energy can also be regulated in response to feedback during treatment. For example, the amount of light being delivered can be regulated based on concurrent analysis of photosensitizing agent penetration, heat level in the tissue (e.g., skin), or the level of discomfort being experienced by the subject.

In one embodiment, the amount or “dose” ranges from about 5 to about 200 J/cm² or in particularly, about 5 to about 10 J/cm², about 10 to about 20 J/cm², about 20 to about 30 J/cm², about 30 to about 40 J/cm² or about 40 to about 50.

In one embodiment, In one embodiment, the present invention provides a method of heat-enabled, low-dose PDT. By “low-dose PDT”, it is meant a total photodynamic therapy experience at substantially lower levels of intensity of the radiation employed and the time of exposure to light (i.e., low-dose light). In one embodiment, the intensity of radiation employed is about 10, about 20, about 30, about 40, about 50, about 60 or about 70% less than prior art methods. In another embodiment, the amount of radiation is less than about 100 J/cm², less than about 80 J/cm², less than about 70 J/cm², or more particularly, less than about 65 J/cm², less than about 60 J/cm², less than about 55 J/cm², less than about 50 J/cm², less than about 45 J/cm², less than about 40 J/cm².

In another embodiment, the amount of radiation is less than about less than about 40 J/cm², or more particularly, the dose ranges from about 35 to about 40 J/cm², or more particularly, about 37 J/cm².

The fluence rate of the light may vary. In one embodiment, the light source has a fluence rate of between about 1 to about 100 mW/cm².

In the case of skin tissue, a sufficient interval of time is preferably provided to allow the photosensitizer to reach the lower levels of skin such as the basal epidermis and/or the papillary dermis, which is the upper layer of the dermis immediately beneath the epidermis. The particular layer of skin targeted can vary depending on the disease being treated. For example, the time interval can be 1 hour, 30 minutes, 10 minutes, 5 minutes, or any other interval within this range. In addition to providing sufficient time for the photosensitizer to penetrate the skin to the desired level, care should also be taken to avoid providing too much time, which can result in passage of significant amounts of the photosensitizing agent beyond the intended site.

Typically, the administration of photodynamic therapy comprises administering one or more sessions of therapy (e.g., one, two, three, four, or more sessions of therapy) to the subject. According to embodiments, a session includes both the administration of a photoreactive compound to a treatment area and the irradiation of the treatment area, which can occur over the course of several hours or one or more days or weeks. According to other embodiments, a session comprises just irradiation of the treatment area alone. In some embodiments, the photodynamic therapy is administered in two sessions. In some embodiments, the photodynamic therapy is administered in only one session.

In exemplary embodiments, the method further comprises monitoring the disease or disorder of the skin following administration of photodynamic therapy according to the present invention wherein a lack of clinical response to the method is indicative that the method should be repeated and/or that the amount of the photosensitive agent and/or the dose of light should be increased. Monitoring may comprise visual inspection, palpation, imaging, assaying the presence, level, or activity of one or more biomarkers associated with the disease or disorder and/or clinical response in a sample obtained from the subject, or a combination of two or more of the foregoing. The monitoring is preferably done regularly, which for the purposes of thus disclosure is at least several times a week and preferably about daily.

Where the disease or disorder is a non-melanoma skin cancer, monitoring may be directed to one of more of the following: tumor size, rate of change in tumor size, appearance of a new tumor (i.e., recurrence), rate of appearance of new tumors (i.e., rate of recurrence), change in a symptom of NMSC, appearance of a new symptom associated with the NMSC, quality of life or a combination of two or more of the foregoing.

In one embodiment, the method of the present invention reduces the recurrence of NMSC relative to the same method in the absence of a pre-heating step. In a particular embodiment, recurrence is reduced by about 5, about 10, about 15, about 20, about 25, about 30, about 35, about 40, about 45 or about 50% or more. In another particular embodiment, recurrence is less than about 10%, less than about 8%, less than about 6%, less than about 4% or less than about 2%. In another particular embodiment, recurrence is about 10, about 9.5, about 9, about 8.5, about 8, about 7.5, about 7, about 6.5, about 6, about 5.5, about 5, about 4.5, about 4, about 3.5, about 3, about 2.5, about 2, about 1.5, about 1.0, or about 0.5 or less. Recurrence may be measured at any suitable time point, such as, for example, about six months, about one year, about two years, about three years, about four years or about 5 years.

Without being bound by any particular theory, the method of the present invention is believed to permit improvement in determining the margin of the tumor. In one embodiment, the ability to determine a tumor margin is about 5, about 10, about 15, about 20, about 25, about 30, about 35, about 40, about 45 or above 50% or more improved using the present method.

In another embodiment, the method of the present invention reduces the time to recurrence of NMSC relative to the same method in the absence of a pre-heating step. In a particular embodiment, time to recurrence is reduced by about 5, about 10, about 15, about 20, about 25, about 30, about 35, about 40, about 45 or about 50% or more. In another particular embodiment, the mean time to recurrence was reduced by about 1 to about 36 months, or more particularly, about 1, about 3, about 6, about 9, about 12, about 15, about 18, about 21, about 24, about 27, about 30, about 33 or about 36 months or more.

Advantageously, the method of the present invention permits improvement in the determinations of tumor margin relative to the same method in the absence of step (i). In exemplary embodiments, the ability to determine the tumor margin is improved about 5, about 10, about 15, about 20, about 25, about 30, about 40, about 45 or about 50% or more.

In exemplary embodiments, the method of the present invention permits treatment with accuracy as to margins consistent with conventional surgical treatment.

Where the disease or disorder is acne, monitoring may be directed to one or more of the following: acne lesion counts, acne lesion severity.

In one embodiment, the method of the present invention produces a decrease in mean acne lesion counts in a subject. In a particular embodiment, the percentage reduction is about 10 or more, about 20 or more, about 30 or more, about 40 or more, about 50 or more, about 60 or more, about 70 or more, about 80 or more, or about 90% or more.

In a particular embodiment, the method of the present invention produces a decrease in mean acne lesion counts in a subject of between about 40% and about 90%, more particularly, about 45% and about 85%, and even more particularly about 47% and about 80%.

The percentage reduction may increase as the method is carried out multiple times. For example, the decrease in mean acne lesions may be about 30% or more after a first treatment, about 40% or more after a second treatment and/or about 50% or more after a third treatment.

The result achieved by the method of the present invention may persist. For example, the decrease in mean acne lesion count may persist for about one, about two, about three, about four, about five, about six, about seven, about eight or about nine months or more. Persistence for about twelve months or more may be considered clinical remission.

In one embodiment, a population of patients treated according to the method of the present invention has a decrease in acne lesion count relative to patients treated according to the same method in the absence of step (i). In a particular embodiment, the acne lesion count in the population treated according to the method of the present invention is decreased by about 5, about 10, about 15, about 20, about 25, about 30, about 35, about 40, about 45, about 50%, about 60%, about 70%, about 80% or about 90% or more relative to patients treated according to the same method in the absence of step (i).

In certain embodiments, the methods reduce acne lesion count in a period of time in the range of one to about twelve weeks. In some embodiments, the methods result in very few instances (e.g. <1%) of redness, dryness, and peeling of treated skin.

In another embodiment, a population of patients treated according to the method of the present invention has a decrease in acne lesion severity relative to patients treated according to the same method in the absence of step (i). In a particular embodiment, the acne lesion severity in the population treated according to the method of the present invention is decreased by about 5, about 10, about 15, about 20, about 25, about 30, about 35, about 40, about 45, about 50%, about 60%, about 70%, about 80%, about 90% or more relative to patients treated according to the same method in the absence of step (i).

The method of the present invention may result in diminished side effects relative to the same method in the absence of a pre-heating step prior to administration of the photosensitive agent. These side effects include, for example, discomfort, scaling, and sterile postulation.

In one embodiment, discomfort is reduced by about 10, about 20, about 30, about 40, about 50, about 60, about 70, about 80 or about 90% or more.

In another embodiment, scaling is reduced by about 10, about 20, about 30, about 40, about 50, about 60, about 70, about 80 or about 90% or more.

In yet another embodiment, sterile postulation is reduced by about 10, about 20, about 30, about 40, about 50, about 60, about 70, about 80 or about 90% or more.

In a particular embodiment, the method of the present invention provides a statistically significant two-point reduction in an Investigator's Global Assessment (IGA) of acne severity compared to vehicle control at six, nine or twelve weeks.

In another particular embodiment, the method of the present invention provides an absolute reduction in the number of acne lesions (non-inflammatory, inflammatory or both) of about 2, about 4, about 6, about 10, about 12, about 14, about 16, about 18, about 20, about 22 or about 24 or more.

In one embodiment, the method of the present invention decreases the risk of atrophic (depressed) acne scars. In a particular embodiment, the risk is reduced by about 10, about 20, about 30, about 40, about 50, about 60, about 70, about 80 or about 90% or more.

### III. Combination Therapy

The present invention includes methods of treatment involving co-administration of one or more therapies in addition to the PDT with prior-heat treatment described above. As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

In exemplary embodiments, the method of the present invention may be used in combination with one or more agents used to treat actinic keratoses. Non-limiting examples of such agents include 5-fluorouracil (5-FU) and imiquimod (Aldara, Zyclara).

In exemplary embodiments, the method of the present invention may be used in combination with administration of one or more anti-cancer agents, such as cytotoxic agents, chemotherapeutic agents, anti-signaling agents, and anti-angiogenic agents.

In one embodiment, the method of the present invention may be used in combination with administration of one or more agents used to treat BCC. Non-limiting examples of such agents include imiquimod (Aldara, Zyclara), flurouracil-topical (Efudex, Carac, Fluoroplex), vismodegib and sonidegib.

In one embodiment, the method of the present invention may be used in combination with administration of one or more agents used to treat SCC Non-limiting examples of such agents include imiquimod (Aldara, Zyclara), flurouracil-topical (Efudex, Carac, Fluoroplex), and cetuximab (Erbitux).

In exemplary embodiments, the methods of the present invention may be used in combination with administration of one or more anti-acne agents. Non-limiting examples of useful anti-acne actives include the keratolytics such as salicylic acid (o-hydroxybenzoic acid), derivatives of salicylic acid such as 5-octanoyl salicylic acid and 4 methoxysalicylic acid; retinoids such as retinoic acid and its derivatives (e.g., cis and trans); sulfur-containing D and L amino acids and their derivatives and salts, particularly their N-acetyl derivatives, a preferred example of which is N-acetyl-L-cysteine; lipoic acid; sebostats such as flavonoids and bioflavonoids; bile salts such as scymnol sulfate and its derivatives, deoxycholate, and cholate; abietic acid; adapalene; allantoin; aloe extracts; arbietic acid and its salts; aryl-2,4 dioxo oxazolidine derivatives; ASEBIOL® (available from Laboratories Serobiologiques, located in Somerville, N.J.); azaleic acid; barberry extracts; bearberry extracts; belamcanda chinensis; benzoquinolinones; benzoyl peroxide; berberine; BIODERMINE® (available from Sederma, located in Brooklyn, N.Y.); bioflavinoids; bisabolol; S-carboxymethyl cysteine; carrot extracts; cassin oil; clove extracts; citral; citronellal; climazole; CREMOGEN® M82 (available from Dragoco, located in Totowa, N.J.); cucumber extracts; dehydroacetic acid and its salts; dehydroeplandersterone salicylate; dichlorophenyl imidazoldioxolan which is commercially available as COMPLETECH MBAC-OS® (from Lipo, located in Paterson, N.J.); DL valine and its esters; DMDM hydantoin; Epicutin TT (available from CLR); erythromycin; escinol; ethyl hexyl monoglyceryl ether; ethyl 2-hydroxy undecanoate; farnesol; farnesol acetate; geranoil; glabridin; gluconic acid; gluconolactone; glyceryl monocaprate; glycolic acid; grapefruit seed extract; gugu lipid; Hederagenin (available from Maruzen); hesperitin; hinokitol; hops extract; hydrogenated rosin; 10 hydroxy decanoic acid; ichtyhol; interleukin 1 alpha antagonists; iodo-2-propynyl butyl carbamate; Kapilarine (available from Greentech); ketoconazole; lactic acid; lemon grass oil; Lichochalcone LR15 (available from Maruzen); linoleic acid; LIPACIDE® C8CO (available from Seppic, located in Paris, France); lovastatin; 4 methoxysalicylic acid; metronidazole; minocycline; mukurossi; neem seed oil; vitamin B.sub.3 compounds (such as niacinamide and nicotinic acid); nisin; 5-octanoly salicylic acid; octopirox; panthenol; 1-pentadecanol; peonia extract; peppermint extract; phelladendron extract; 2-phenyl-benzothiophene derivatives; phloretin; PHLOROGINE® (available from Secma); phosphatidyl choline; proteolytic enzymes; quercetin; red sandalwood extract; resorcinol; rosemary extract; rutin; sage extract; salicin; salicylic acid; skull cap extract; siber hegner extract; siberian saxifrage extract; silicol; sodium lauryl sulfate; sodium sulfoacetamide; Sophora Extract (available from Maruzen); sorbic acid; sulfur; sunder vati extract; tea tree oil; tetracyline; tetra hydroabietic acid; thyme extract; tioxolone; tocopherol; trehalose 6-undecylenoate; 3 tridecene-2-ol; triclosan; tropolone; UNITRIENOL® T27 (available from Unichem, located in Gouda, Netherlands); vitamin D.sub.3 and its analogs; white thyme oil; willow bark extract; wogonin; Ylang Ylang; zinc glycerolate; zinc linoleate; zinc oxide; zinc pyrithione; zinc sulfate and mixtures thereof.

In one embodiment, the methods of the present invention may be used in combination with administration of one or more “anti-inflammatory” compounds. Representative, non-limiting examples of anti-inflammatory compounds include azelaic acid, clindamycin, niacinamide, and tretinoin.

### EXAMPLES

### Example 1: Treatment of Inflammatory and Cystic Acne

A total of six (6) patients were treated. The affected area was treated for about one (1) hour with a warming mask at 40° C., followed by application of 10% ALA gel and 37 J/cm² of red light at 630 nm.

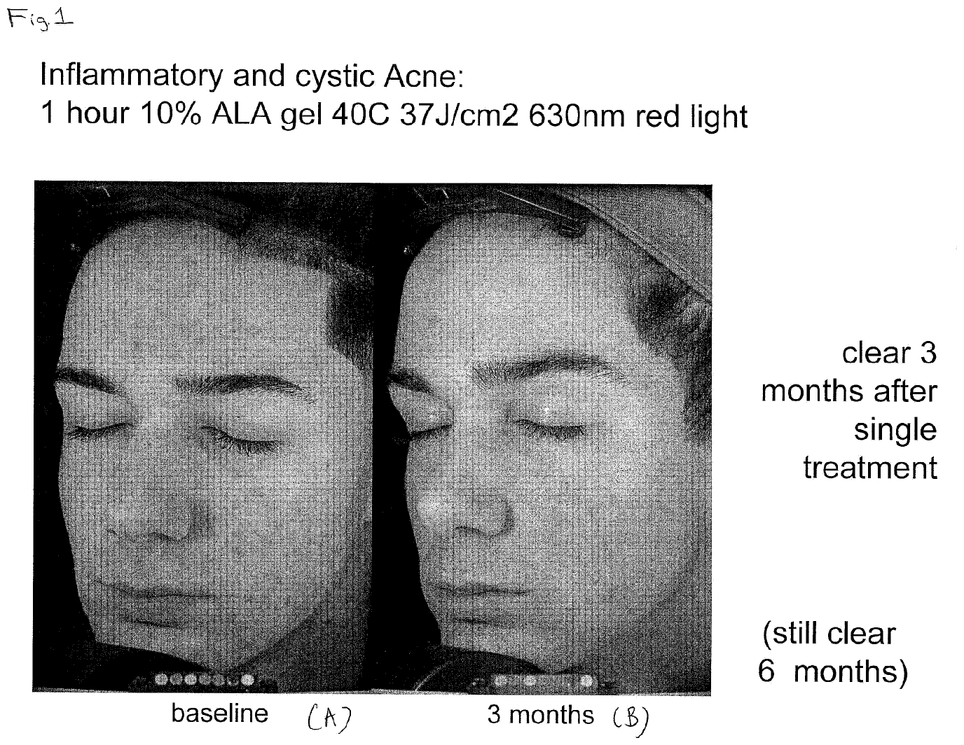

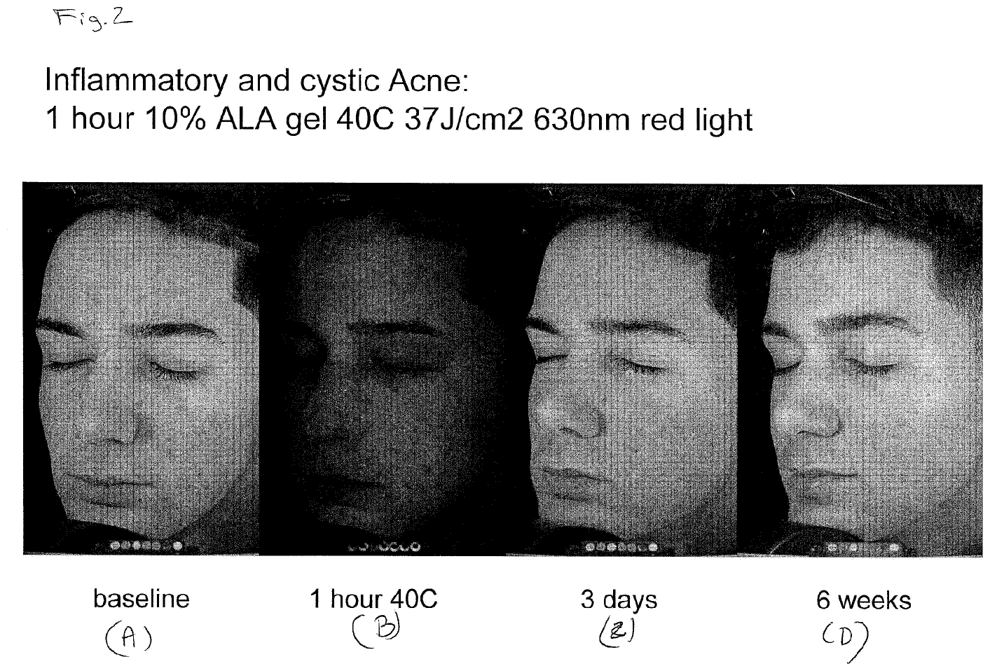

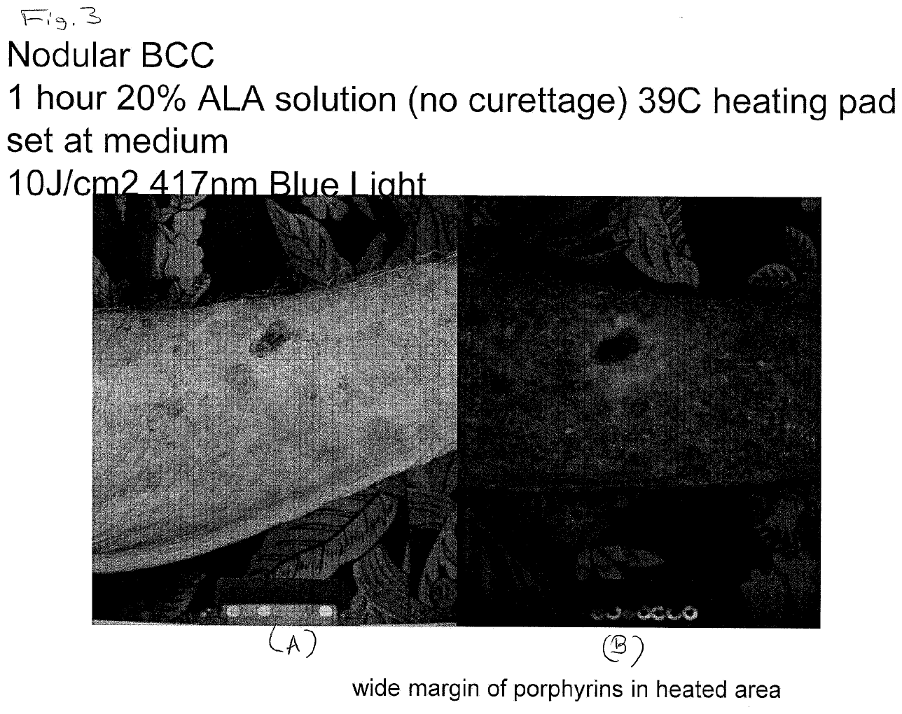

The results included a mild, sun-burn like reaction, followed by pustular eruption at day 3 and 95% clear at one (1) month. The result was sustained for at least six (6) months. See FIG. 1, which shows baseline (FIG. 1A) and results (clear) at three (3) months (FIG. 1B). See also FIG. 2, which shows results at baseline (FIG. 2A), 1 hour (using poryphorin camera)(FIG. 2B), three (3) days (FIG. 3B) and six (6) weeks.

### Example 2: Treatment of Basal Cell Carcinoma on Extremities

Patients with nodular basal cell carcinoma were treated. The affected area was treated with a heating pad at 39° C. Each was also treated with 20% ALA gel without curettage. Blue light at 37 J/cm² was applied at 410 nm.

A wide margin of poryphorins was detected. See FIG. 3, including FIG. 3B which shows a wide margin of pophyrins in the heated area.

### Example 3: Treatment of Basal Cell Carcinoma on Extremities

Patients with recurrent nodular and infiltrative basal cell carcinoma were treated. The affected area was treated with a heating pad at 40° C. Each was also treated for one (1) hour with 10% ALA gel following curettage. Red light at 630 nm of 37 J/cm² was applied.

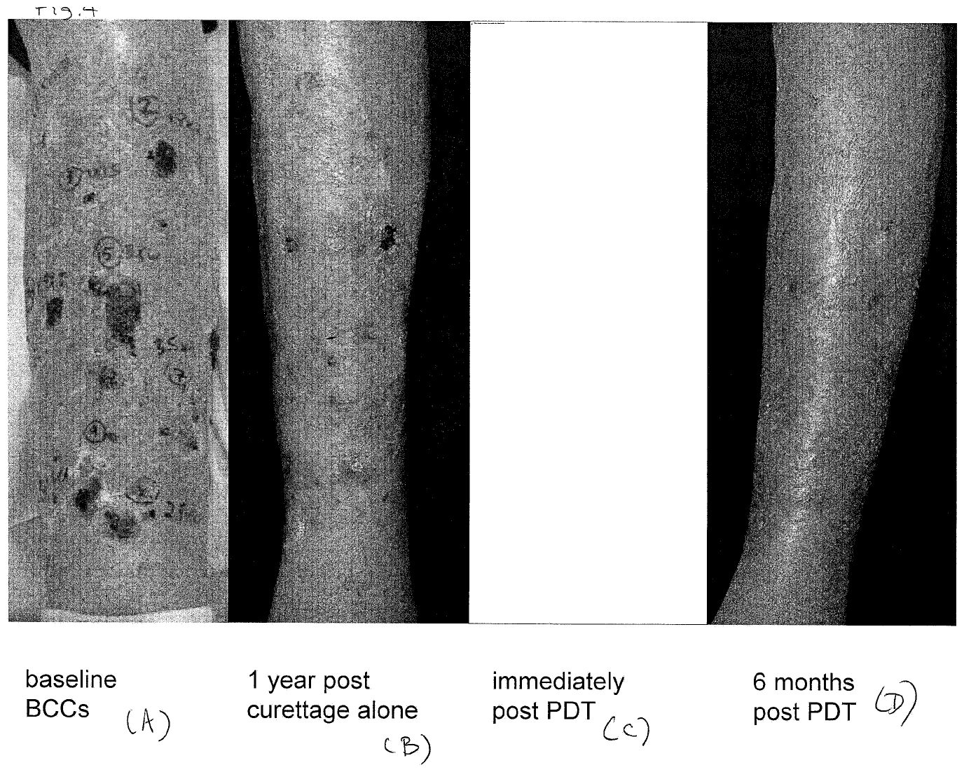

Results indicate that patients were clear for at least six (6) months with a single treatment. See FIG. 4, which shows baseline (FIG. 4A), one (1) year following post curettage alone (FIG. 4C) and six (6) months following treatment with PDT (FIG. 4D).

### Example 4: Treatment of BCC on Scalp

Patients with infiltrative basal cell carcinoma of the scalp were treated. The affected area was heat treating using a 40° C. heating pad. A 20% ALA solution was applied for one hour, followed by 10 J/cm² of blue light at 417 nm.

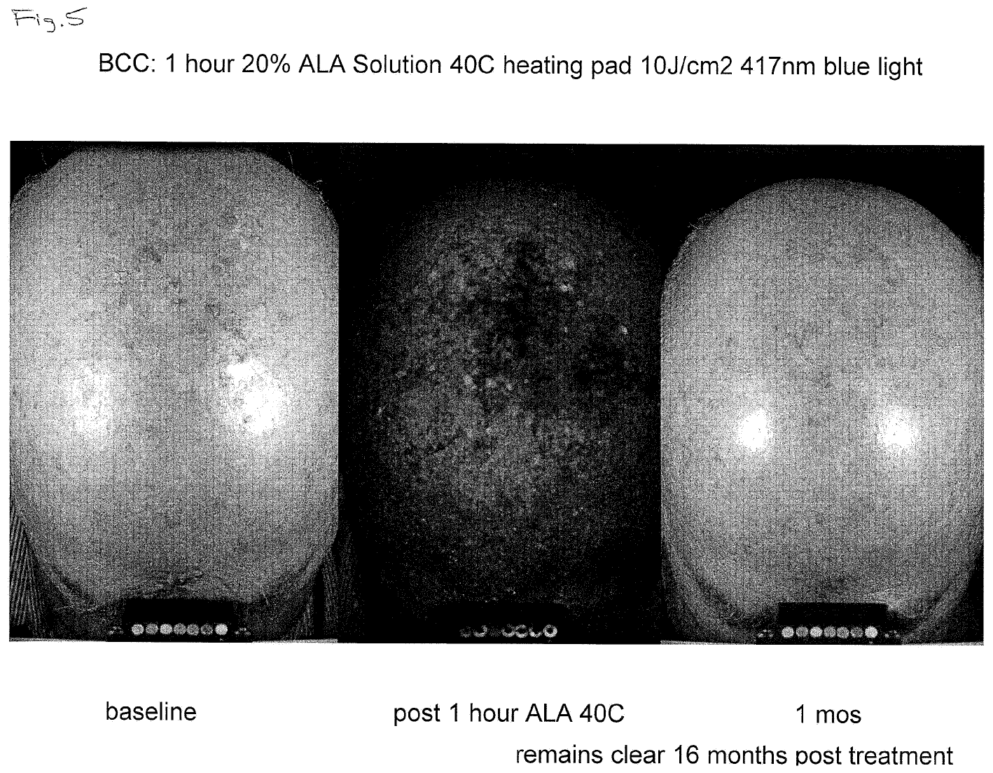

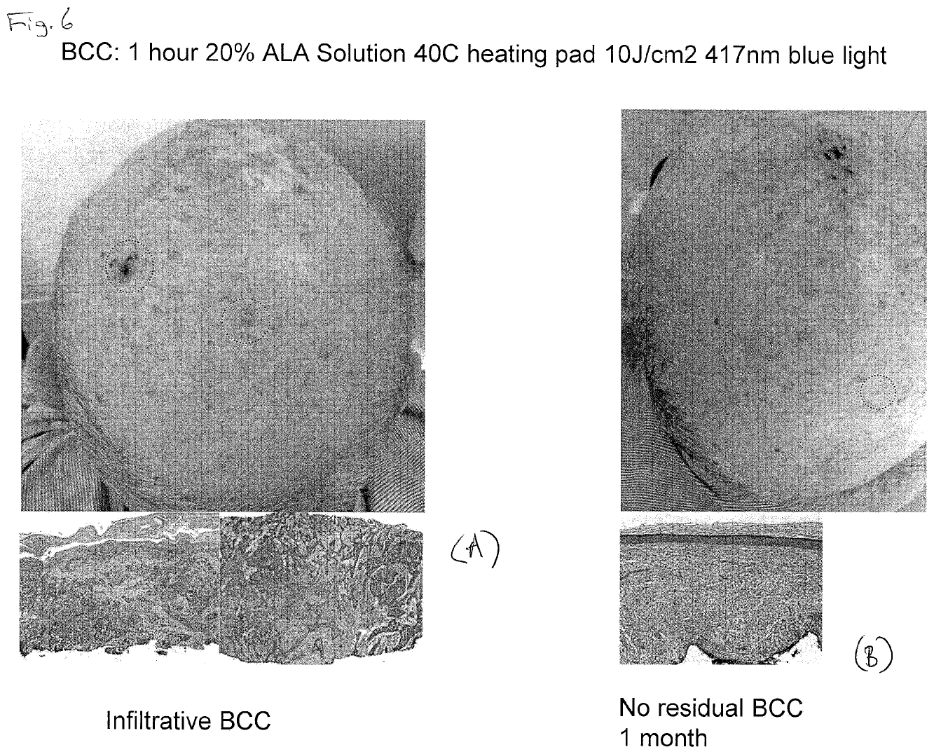

Biopsy showed that lesions were clear one month following treatment (FIG. 6B). Additional evidence shows lesions were clear up at one month (FIG. 5), which persisted for sixteen (16) months following treatment.

### Example 5: Treatment of SCC-IS

Patients with SCC-IS were treated. The affected area was heat treated with a sodium acetate warming pad to 40° C. A 20% ALA solution was applied to each for 20 minutes. Blue light was applied at 417 nm for 10 J/cm².

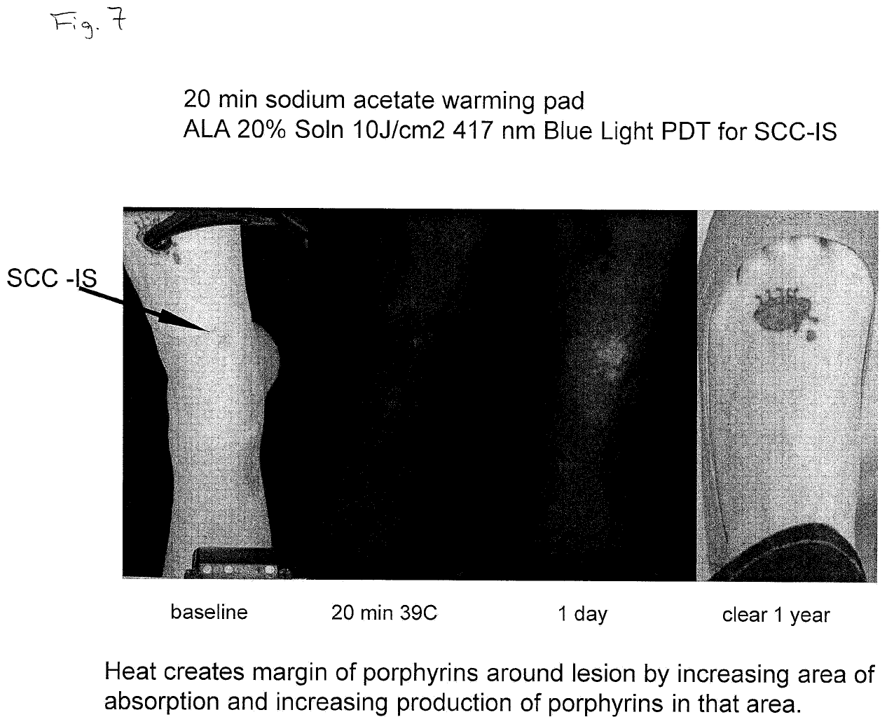

The results indicate that each was clear one (1) year following a single treatment. See FIG. 7.

### Example 6: Treatment of Refractory Disseminated Porokeratosis with Related SCC

Patients with refractory disseminated porokeratosis with related SCC. The affected area was heat treated with a 40° C. heating pad. 10% ALA gel was applied for one (1) hour. Red light was applied with an energy of 37 J/cm2 and a wavelength of 630 nm.

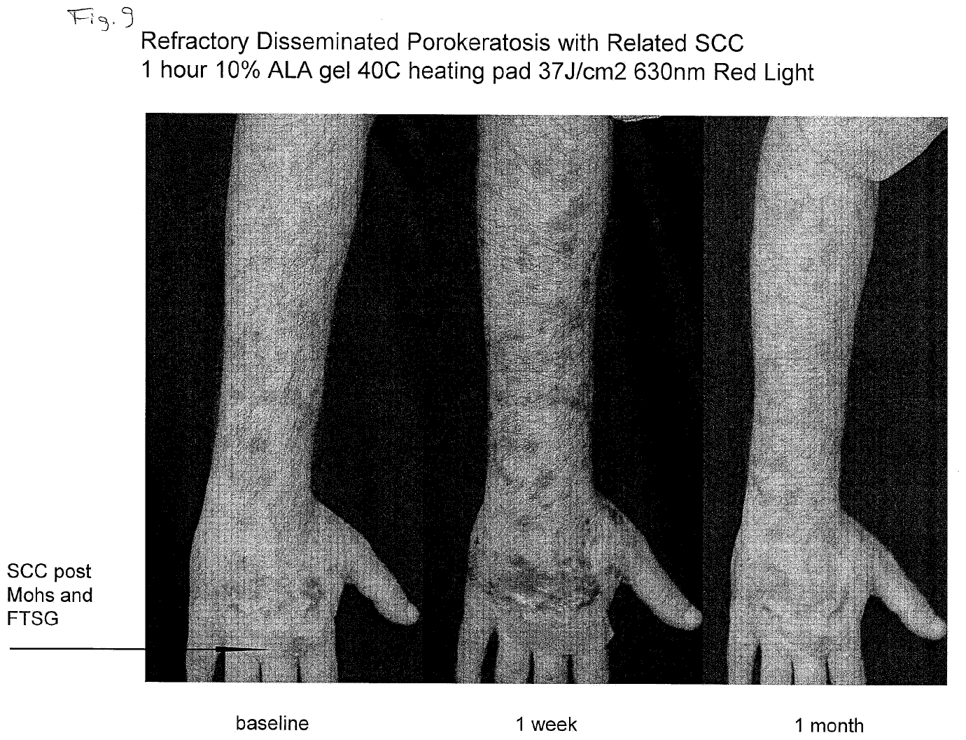

Results are shown in FIG. 9, including baseline (FIG. 9A), one (1) week (FIG. 9B) and one (1) month (FIG. 9C).

### Example 7: Treatment of Actinic Keratosis

Patients with actinic keratosis were treated. Heat was applied to the affected area using a 40° C. warming mask. A 20% ALA solution was applied, following 20 minute incubation. Light was then applied.

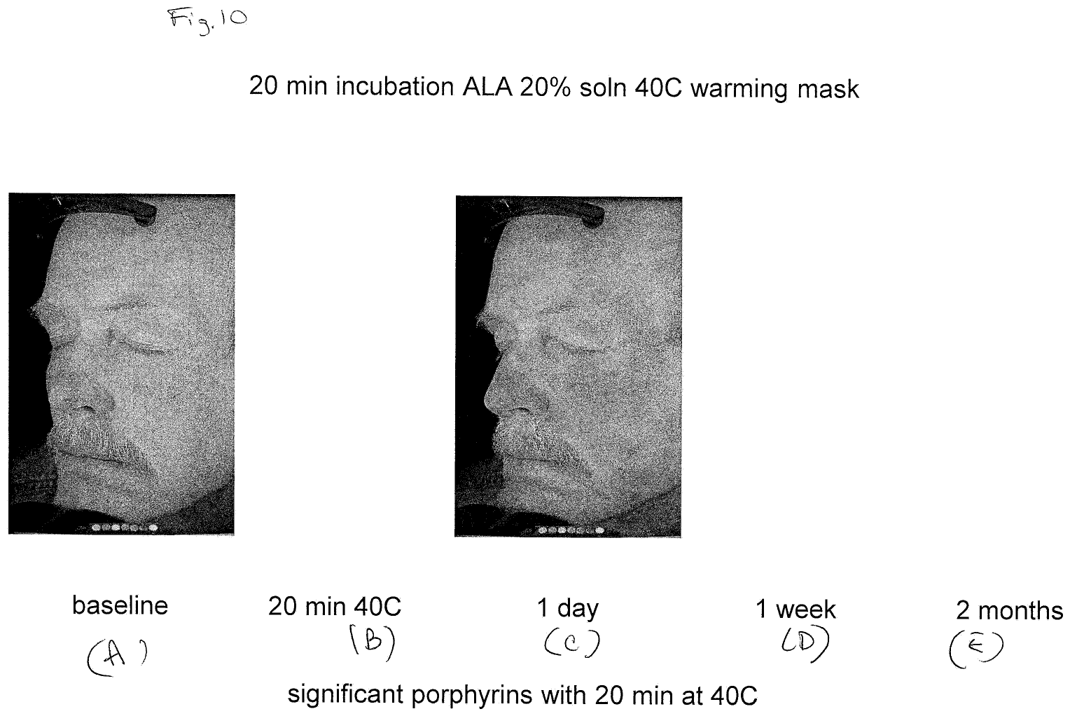

Results are shown in FIG. 10, including baseline (FIG. 10A), twenty (20) minutes (FIG. 10B), one (1) day (FIG. 10C), one (1) week (FIG. 10D) and two (2) months (FIG. 10E). Significant porphyrins are shown.

### Example 8: Treatment of Actinic Keratosis

Heat was applied to the affected area using a 40° C. warming mask. A 20% ALA solution was applied, following 30 minute incubation. Light was then applied.

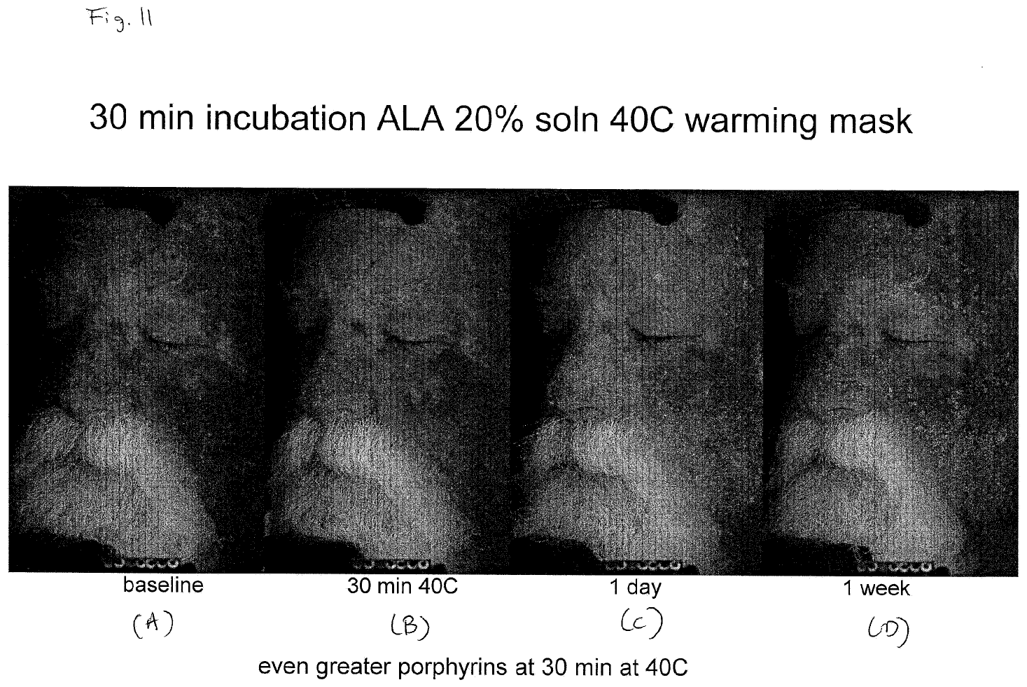

Results are shown in FIG. 11, including baseline (FIG. 11A), thirty (30) minutes (FIG. 11B), one (1) day (FIG. 11C) and one (1) week (FIG. 11D). Even greater porphyrins are shown.

### Example 9: Treatment of Actinic Keratosis

A 20% ALA solution was applied for one hour (room temperature 70 F), following a 60 minute incubation. Light was then applied.

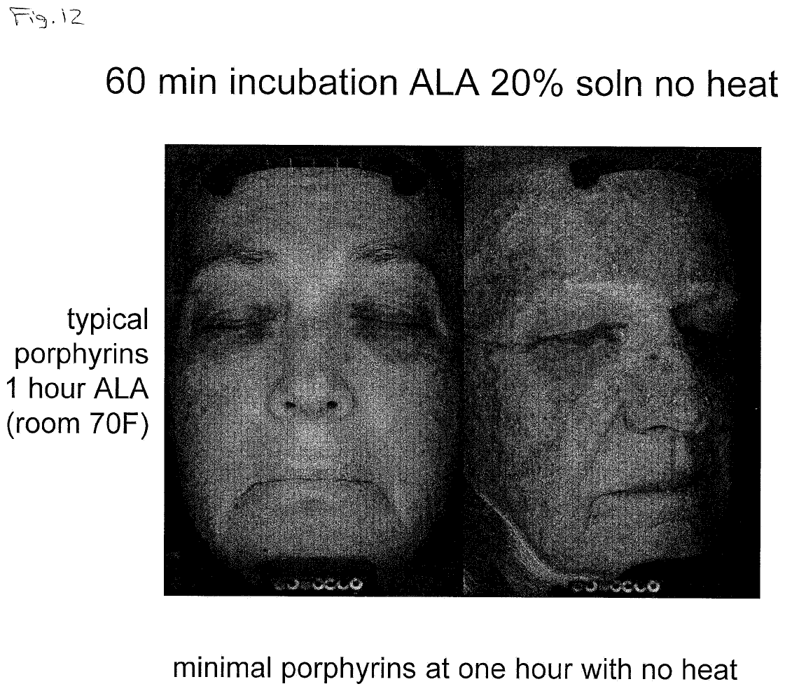

Results are shown in FIG. 12, including baseline (FIG. 12A) and one (1) hour. Minimal porphyrins are shown with no heat.

### Example 10: Treatment of Moderate Inflammatory and Pustular Acne

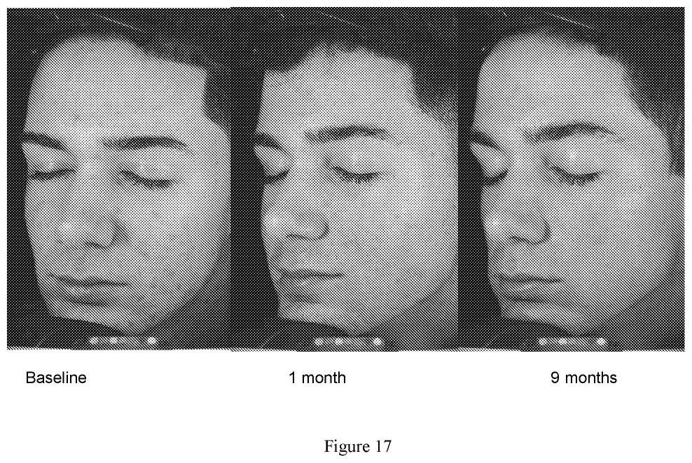

A one hour incubation with 10% aminolevulinic acid (ALA) with a sodium acetate warming mask (skin temperature 40 C) followed by 37 J/cm2 635 nm red light was used to treat an index patient with moderate inflammatory and pustular acne on the face. The reduction in lesion counts was persistent 9 months after a single thermal PDT, as shown in FIG. 17 and Table II.

TABLE I
Inflammatory and Non-Inflammatory Lesion Counts and
Clearance Rates Determined by a Blinded Investigator:

| | | Non- | Total | Percent |
|---|---|---|---|---|
| Visit | Inflammatory | Inflammatory | Lesions | Clearance |
| baseline | 31 | 22 | 53 | |
| 1 month | 14 | 7 | 21 | 60 |
| 3 months | 10 | 13 | 23 | 57 |
| 9 months | 6 | 4 | 10 | 81 |

### Example 11: Treatment of Moderate Inflammatory and Pustular Acne

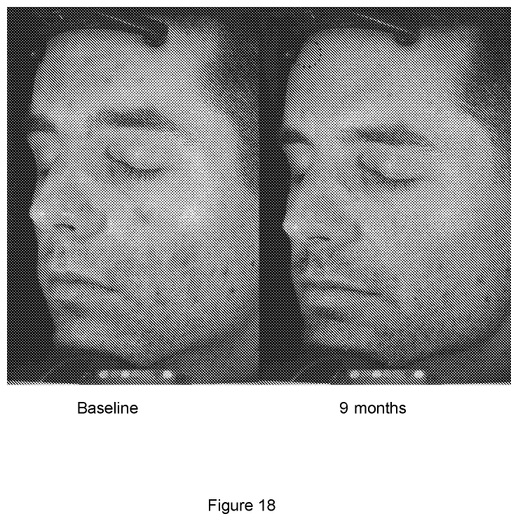

A patient with moderate inflammatory and pustular acne on the face using a one hour incubation with 10% aminolevulinic acid with a sodium acetate warming mask (skin temperature 40° C.) followed by 37 J/cm2 635 nm red light. Remodeling of acne scars is shown 9 months after single thermal PDT, as shown in FIG. 18 and Table II.

TABLE II

| | Inflammatory Lesions | |
|---|---|---|
| Visit | with Potential to Scar | Scars Present |
| baseline | 94 | — |
| 9 months | 8 | 1 |

### Example 12: Treatment of Actinic Keratoses

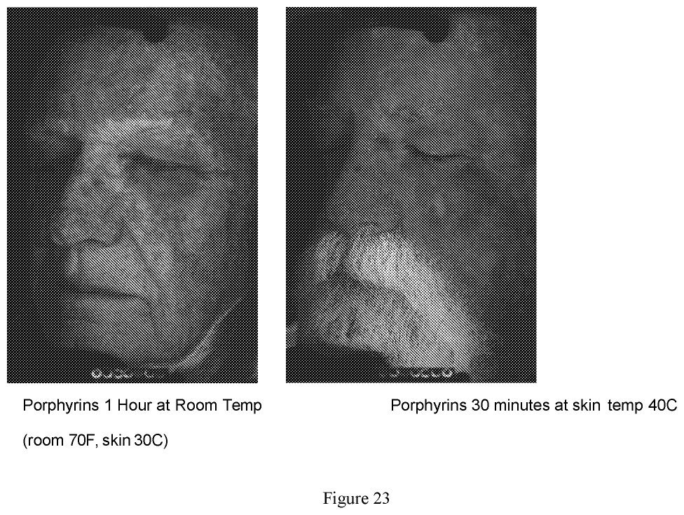

Facial skin with actinic keratoses was treated with a 30 minute incubation with 20% ALA at 40 C using a sodium acetate warming mask compared to a one hour incubation at room temperature (room temperature 70° F., skin temperature 30° C.). Increased porphyrin was observed, as shown in FIG. 23. Porphyrin images were captured using the VISIA-CR® 4.1 camera system and analyzed using the Image-Pro® Plus 7.0 (IPP®, Media Cybernetics Inc.) imaging analysis software. The intensity of PpIX fluorescence signals measured within the masked area was quantified on a scale of 0 to 255.

TABLE III

| | 1 Hour Incubation 30 C. | 30 Minutes 40 C. |
|---|---|---|
| Mean Porphyrin Intensity | 71.5402 | 131.2849 |

1. A method of treating facial acne in a subject in need thereof, comprising (i) administering heat to an affected area of the subject's skin using a thermal delivery device to achieve a temperature of between about 38 and about 42° C. for a suitable time; (ii) incubating a pharmaceutical composition comprising at least one photosensitizing agent for an incubation period of less than about 14 hours to provide an incubated pharmaceutical composition; (iii) administering a therapeutically effective dose of the incubated pharmaceutical composition to the affected area for a suitable time period; (iii) administering a suitable dose of red light to the affected area, thereby treating the facial acne.

2. The method of claim 1, wherein the incubation time is less than about 10 hours.

3. (canceled)

4. (canceled)

5. The method of claim 1, wherein heat is applied using a sodium acetate mask.

6. The method of claim 1, wherein the wherein the heat is administered in (i) for about 60 minutes.

7. The method of claim 1, wherein the at least one photosensitizing agent is 5-ALA.

8. The method of claim 1, wherein the pharmaceutical composition is a gel.

9. The method of claim 1, wherein the pharmaceutical composition is a nano-emulsion.

10. The method of claim 7, wherein the 5-ALA is present at about 20 wt. % of the pharmaceutical composition.

11. The method of claim 7, wherein the 5-ALA is present at about 10 wt. % of the pharmaceutical composition.

12. The method of claim 1, wherein the wavelength of the red light is between about 625 and about 645 nm.

13. (canceled)

14. The method of claim 1, wherein the dose of the red light is about 37 J/cm².

15. The method of claim 1, wherein the wavelength is about 630 nm at a distance of 6 cm from the subject's skin.

16. The method of claim 1, wherein the acne lesion count is reduced by greater than about 30% as a result of treatment.

17. (canceled)

18. (canceled)

19. The method of claim 16, wherein the reduction persists for at least about three months.

20. (canceled)

21. (canceled)

22. The method of claim 1, wherein treatment produces less scaling than the same method not employing step (i).

23. The method of claim 1, wherein treatment produces less sterile postulation than the same method not employing step (i).

24. The method of claim 1, wherein the acne is mild acne.

25. The method of claim 1, wherein the acne is moderate acne.

26. The method of claim 1, wherein the acne is severe acne.

27. The method of claim 1, wherein the acne is inflammatory acne.
